(19) United States
(12) Patent Application Publication
(10) Pub. No.: US 2024/0006067 A1
(43) Pub. Date: Jan. 4, 2024

(54) SYSTEM BY WHICH PATIENTS RECEIVING TREATMENT AND AT RISK FOR IATROGENIC CYTOKINE RELEASE SYNDROME ARE SAFELY MONITORED

(71) Applicant: BIOSIGNS PTE. LTD., Jurong East (SG)

(72) Inventors: Michael Joseph Pettinati, Atlanta, GA (US); Nandakumar Selvaraj, San Jose, CA (US)

(21) Appl. No.: 18/341,750

(22) Filed: Jun. 26, 2023

Related U.S. Application Data
(60) Provisional application No. 63/356,987, filed on Jun. 29, 2022.

(51) Int. Cl. G16H 50/20; G16H 50/30; G16H 10/60; G16H 40/20

(52) U.S. Cl. CPC G16H 50/20; G16H 50/30; G16H 10/60; G16H 40/20; G16H 40/67

(57) ABSTRACT

In some examples, a patient care pathway is coupled with a cytokine release syndrome (CRS) prediction system. A CRS prediction machine learning model is used to analyze patient-related health data associated with a monitored user, such as a patient. The health data includes physiological data obtained from sensor devices associated with the monitored patient and user-provided data associated with the monitored patient. A CRS event prediction indicates the probability of an occurrence of a CRS event within a time-period after the prediction is generated. A CRS event that is predicted or detected in progress is graded to indicate a predicted severity. An outcome can also be generated indicating whether the patient's condition is predicted to improve within the future time-period, enabling more accurate early detection of CRS events for improved patient outcomes. In some examples, the prediction can facilitate a patient care pathway for improved, safer, and more cost-effective care.

Cytokine release syndrome (CRS) is a noninfectious systemic inflammatory response syndrome (SIRS). This condition can be a principle severe adverse event (SAE) common in oncology patients treated with immunotherapies. If detected, CRS can be treated to mitigate symptoms and improve patient outcomes. However, symptoms associated with CRS, such as malaise, fever, hypoxia, and hypotension are common to many conditions, complicating early detection and diagnosis by human clinicians. Further, the conditions that share symptoms with CRS require novel treatments and care. Thus, accurate monitoring and timely prediction of CRS and its severity remain an important and necessary challenge to overcome. Patients at risk of developing CRS conditions require continuous monitoring to detect onset of the CRS condition in advance, assess the severity of the condition, and to aid them in seeking immediate clinical attention to plan a course of treatment.

### SUMMARY

The present disclosure relates to cytokine release syndrome (CRS) event prediction coupled with a patient care pathway for patients, such as those at-risk for iatrogenic CRS. The system predicts cytokine release syndrome's onset, undertaking associated severity and deterioration monitoring and prediction, and generates CRS-related notifications through a CRS prediction manager system. Patient-related health data for a monitored patient is obtained. The patient-related health data comprises physiological data obtained from a set of sensor devices associated with the monitored patient and user-provided data, including data provided by the patient, any associated patient caregivers, electronic health records, and/or data provided by the clinician associated with the monitored patient. The patient-related data is analyzed using a trained CRS prediction manager system, including one or more trained machine learning prediction models. A CRS event prediction is generated based on the analysis. The prediction includes a probability of a CRS event occurring within a predetermined time-period after generation of the CRS prediction. Multiple predetermined time-periods could be considered at a given time. A notification is provided of the predicted CRS event if the probability of the CRS event exceeds a threshold probability indicating onset of the CRS event is likely to occur within the predetermined time-period. If a CRS event is predicted to onset, the gradation of that CRS event and the probability the patient may further deteriorate within some window of time can also be included in the generated alert, notification, and or report.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an exemplary block diagram illustrating a system for predicting the onset of, grading, and deterioration of a patient at risk for cytokine release syndrome (CRS) events.

FIG. 2 is an exemplary block diagram illustrating a system for generating CRS event predictions using machine learning (ML) models.

FIG. 3 is an exemplary block diagram illustrating a computing device 300 for CRS onset prediction and event grading.

FIG. 4 is an exemplary block diagram illustrating a CRS prediction manager system for generating CRS event alerts relating to probability of onset or deterioration and the grade of severity.

FIG. 5 is an exemplary block diagram illustrating a CRS prediction manager system including a CRS deterioration algorithm for predicting and alerting patients and caregivers regarding a patient's likelihood to deteriorate following CRS onset.

FIG. 6 is an exemplary block diagram illustrating a CRS prediction manager system including a CRS grading algorithm for generating a predicted grade for a CRS event following CRS onset.

FIG. 7 is an exemplary is an exemplary block diagram illustrating a CRS prediction manager system including a CRS onset algorithm for predicting CRS onset alert.

FIG. 8 is an exemplary flowchart illustrating the process flow of CRS prediction.

FIG. 9 is an exemplary flowchart illustrating the process flow of generating a CRS prediction.

FIG. 10 is an exemplary flowchart illustrating the process flow of generating a CRS event prediction with a predicted grade.

FIG. 11 is an exemplary summary of ground truth rules used to extract and label a patient day during which the patient is experiencing CRS of mild severity.

FIG. 12 is an exemplary summary of ground truth rules used to extract and label a patient day during which the patient is experiencing severe CRS.

FIG. 13 is an exemplary summary of ground truth rules used to extract and label a patient day during which the patient is experiencing no CRS.

FIG. 14 is an exemplary table illustrating a summary of unique patients' days and CRS severity conditions identified and or extracted from a dataset.

FIG. 15 is an exemplary table illustrating AUROC statistics for various models incorporating various input feature sets and separating CRS grades or gradations.

FIG. 16 is an exemplary graph illustrating all feature ROC curves for an exemplary first patient cohort.

FIG. 17 is an exemplary graph illustrating all feature ROC curves for an exemplary second cohort.

FIG. 18 is an exemplary XGBoost feature importance graph illustrating the most predictive features for grading CRS in one example patient cohort when numerous feature types were incorporated as input.

FIG. 19 is an exemplary XGBoost feature importance graph illustrating the most predictive features for grading CRS in one example patient cohort when only vital signs were incorporated as input.

FIG. 20 is an exemplary table illustrating transition matrix data for daily patient CRS grades showing patient CRS grades from a current day to the next.

FIG. 21 is an exemplary line graph illustrating exemplary visualizations of a single patient's journey where ML models predict daily CRS gradation levels and predict relative change in CRS gradations.

FIG. 22 is an exemplary bar graph illustrating exemplary visualizations of a single patient's journey where ML models predict daily CRS gradation levels or predict relative change in CRS gradations.

FIG. 23 is an exemplary graph illustrating ROC performances of ML models predicting relative changes in CRS gradations when incorporating numerous input features.

FIG. 24 is an exemplary graph illustrating ROC performances of ML models predicting five classes of relative changes in CRS gradations when incorporating vital signs.

FIG. 25 is an exemplary illustration of feature importance from ML models predicting five classes of relative change in CRS gradations.

FIG. 26 is an exemplary flowchart showing an improved patient care pathway involving the CRS onset, grading, and deterioration prediction system.

Corresponding reference characters indicate corresponding parts throughout the drawings.

### DETAILED DESCRIPTION

A more detailed understanding can be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that can in isolation and out of context be read as absolute and therefore limiting, can only properly be read as being constructively preceded by a clause such as “In at least some examples, . . . ” For brevity and clarity of presentation, this implied leading clause is not repeated ad nauseum.

Infection and injury, as well as certain immunotherapies, can trigger amplified inflammatory responses of the human body known as Systemic Inflammatory Response Syndrome (SIRS). SIRS is associated with common symptoms and signs including fever, hypothermia, tachycardia, tachypnea, leukocytosis and/or leucopoenia. The definition for SIRS is overly sensitive in acute-care settings. Cytokine Release Syndrome (CRS) is a serious noninfectious SIRS condition that can develop rapidly following certain types of immunotherapy, cancer treatments and therapies used to treat viral infections, pathogens, autoimmune conditions, and monogenic disorders. CRS is often studied as a side effect of cancer treatments, especially immunotherapies.

During treatment of neoplasms (tumors), generalized activation of patient immune system can lead to CRS. CRS can be a severe adverse event of various immunotherapies resulting in rapid onset of elevated levels of cytokines affecting patient organs. Severe cases of CRS can lead to multiple organ failure. However, timely immunosuppression can minimize the CRS events and maximize the benefits of available immunotherapies.

It is critical to detect the development and severity of CRS and prompt for timely clinical attention and intervention. The healthcare costs and likelihood of clinical complications multiply as patients deteriorate to severe stages of CRS. A non-trivial number of patients reach these severe stages. For example, almost half of patients in early CAR-T cell treatment trials required intensive care management following infusion. Thus, time is of the essence when it comes to accurate and specific CRS detection.

Referring to the figures, examples of the disclosure enable a cytokine release syndrome (CRS) prediction manager system that analyzes input data associated with a patient to predict onset of CRS, grade the predicted CRS events and/or predict CRS event outcomes (e.g., deterioration) for a predetermined time-period after the predicted CRS event and/or a predetermined time-period after prediction generation. In some examples, the CRS prediction manager system generates CRS predictions for a twenty-four-hour time period following the time of the prediction generation for increased diagnostic accuracy and improved patient outcomes.

Aspects of the disclosure further enable a patient monitoring system and automated artificial intelligence algorithms that can detect CRS onset or early stages of CRS well in advance and allow for early intervention to produce better clinical and therapeutic outcomes with lower healthcare delivery costs. These artificial intelligence algorithms are further coupled with a patient care pathway to ensure patient safety throughout their treatment.

Some examples include trained machine learning (ML) models that analyze patient-related data, including patient questionnaire responses and real-time vital signs data, to predict onset and severity of CRS in patients at predetermined time-periods after surgical treatment of a neoplasm. This enables faster diagnosis and treatment of CRS with improved accuracy.

Other examples provide predictive results in a visualization, such as charts, graphs, tables, or other visualized data via a user input (UI) device or other input/output (I/O) device. This provides improved user efficiency via UI interaction. These tools enable the caregivers of patients who are triaged to be of sufficiently low risk to be monitored in remote settings to make informed and rapid decisions regarding the need to bring patients back to hospital for treatment, reduce patient burden and lower monitoring requirements, etc.

The CRS prediction manager system monitors and analyzes patient physiological data, including vital signs, using ML model(s) to accurately predict CRS risks and CRS events in advance prior to actual CRS onset as well as predicting CRS symptom progression. In some examples, the CRS prediction manager system dynamically predicts CRS risks and potential outcomes each day based on real-time patient vital signs data. This daily CRS risk prediction enables early detection of CRS and improves clinical outcomes for patients while reducing diagnostic errors. In other examples, there could be an extremely heterogeneous adverse event profile for a treatment. In these examples, CRS risk is predicted before the patient receives a treatment to help caregivers triage patients in need of closer monitoring following infusion using patient-related health history, demographic, laboratory, and genomic data.

The ML-based CRS prediction manager system offers earlier insights into SIRS conditions using data that is readily available in clinical and other patient-treatment and monitoring environments including remote settings. Patients deemed to be at risk or experiencing CRS and SIRS-related events are identified, graded, and assessed as to whether or not they are likely to deteriorate. Patients who are not experiencing SIRS are monitored for potential onset. Patients who are experiencing non-CRS-related SIRS conditions are assessed and monitored using alternative algorithms. This stratification of SIRS patients is done via expert input or algorithmic classification depending on a defined CRS guideline standard. The system allows for monitoring across different patient environments including remote settings for increased flexibility and scalability across patient populations using models that incorporate different data inputs. These inputs include, but are not restricted to, vital signs, clinical scoring input, patient-reported quality of life (QOL) outcomes and symptoms, laboratory measurements, etc. The CRS prediction manager utilizes ML algorithms to predict the CRS grades in advance using common vital signs and clinical scores obtained in oncology patients receiving standard of care treatments.

The conventional computing device operates in an unconventional manner by predicting onset and event grading of CRS in patients during a predetermined time-period in oncology patients treated for neoplasms using passively and or actively input patient-related health data. In this manner, the computing device is used in an unconventional manner and allows faster and more efficient CRS diagnosis and treatment for improved patient outcomes, thereby improving the functioning of the underlying computing device. The utilization of the computing device in an unconventional manner allows for the implementation of an improved care pathway that protects patients receiving treatments that can cause iatrogenic CRS. This care pathway protects patients from when they are considered for the treatment through the point at which they are discharged from monitoring following treatment.

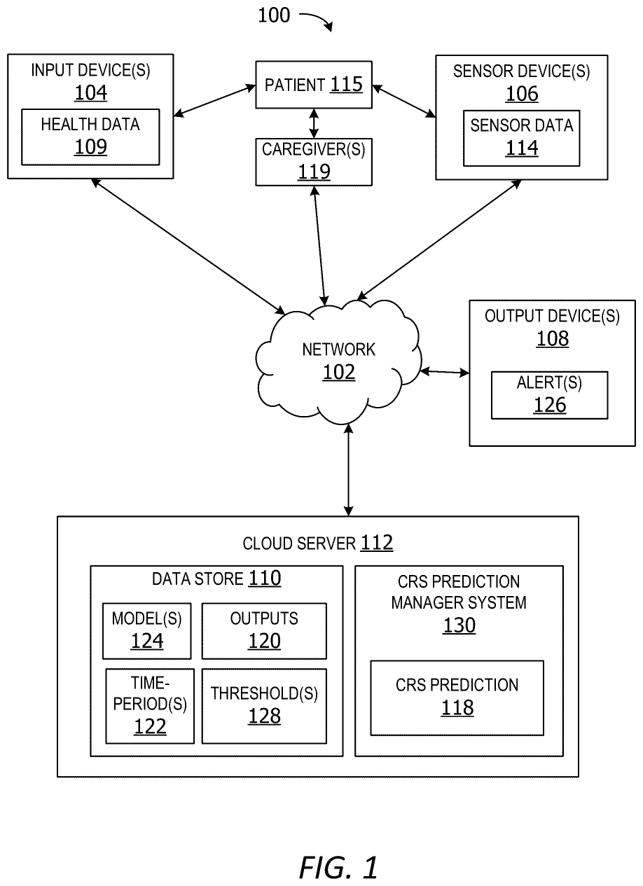

Referring again to FIG. 1, an exemplary block diagram illustrates a system 100 for predicting the onset of, grading, and deterioration of a patient at risk for cytokine release syndrome (CRS) events. In some examples, the system includes a network 102 enabling data transmission between devices and/or other systems connected to the network 102, such as, but not limited to, one or more input device(s) 104, sensor device(s) 106, output device(s) 108, a remote data store and/or a cloud server, such as, but not limited to, the cloud server 112.

Network 102, in some examples, is implemented by one or more physical network components, such as, but without limitation, routers, switches, network interface cards (NICs), and other network devices. The network 102 is any type of network for enabling communications with remote computing devices, such as, but not limited to, a local area network (LAN), a subnet, a wide area network (WAN), a wireless (Wi-Fi) network, or any other type of network. In this example, network 102 is a WAN, such as the Internet. However, in other examples, the network 102 is a local or private LAN.

The patient input device(s) 104 represent any device executing computer-executable instructions. The patient input device(s) 104 can be implemented as a mobile computing device, such as, but not limited to, a wearable computing device, a mobile telephone, laptop, tablet, computing pad, netbook, gaming device, and/or any other portable device. The patient input device(s) 104 include at least one processor and a memory (not shown). The patient input device(s) 104 can also include a user interface component (not shown). A patient 115 associated with the patient input device(s) 104 uses the patient input device(s) 104 to provide health data 109. In some cases, where patient 115 is unable to provide the health data, a family member or other caregiver may utilize the patient input device(s) 104 to provide the patient health data 109. In some examples, health data 109 includes user-provided responses to one or more questions in a questionnaire.

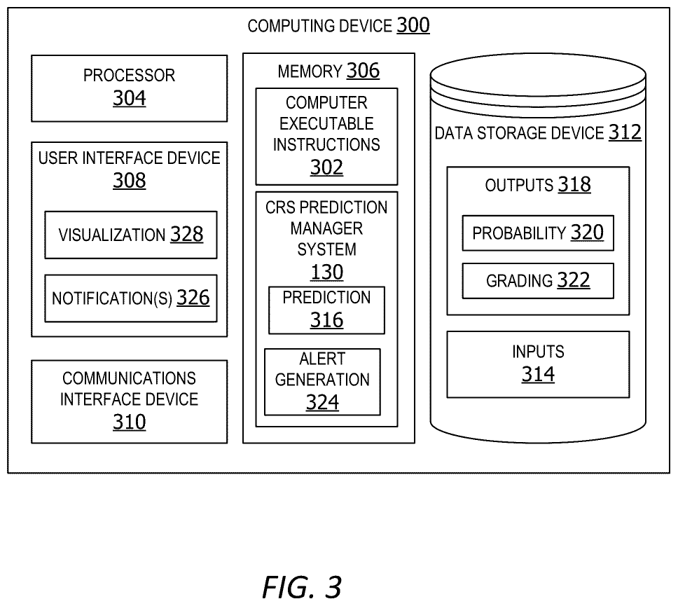

The user-provided data in the patient-related health data 109 can include patient feedback provided by patient 115, such as input symptoms and patient reported outcomes (PROs) and/or questionnaire responses. Other user-provided data is provided by care team members, such as one or more caregiver(s) 119 (clinician or another caregiver), monitoring the patient. The user-provided data can include Glasgow coma scale (GCS) data, AVPU (alert, voice, pain, unresponsive), or other conscious state assessment input by the clinician. In some examples, the caregiver(s) 119 interacts with the cloud system via a computing device, such as, but not limited to, a computing device, the one or more sensor device(s) 106 including a communications interface device, or any other type of device enabling the caregiver(s) 119 to interact with the cloud system. In some examples, the computing device is any type of device executing computer-executable instructions, such as, but not limited to, the computing device 300 in FIG. 3 below. The computing device can include a mobile computing device, such as a tablet or smart phone.

The sensor device(s) 106 includes one or more sensor devices for generating sensor data 114 associated with patient 115. The sensor device(s) 106 in some examples include passive devices for generating sensor data 114, such as wearable sensor devices worn by the patient 115.

In this example, the user is a patient receiving treatment in a clinical setting. However, in other examples, patient 115 is remote from a clinical setting, such as in an out-patient, rehabilitation, home, or other setting. The sensor device(s) 106 include wearable sensor devices gathering continuous sensor data and/or sensor devices which are periodically used to obtain episodic sensor data. The sensor device(s), in some non-limiting examples, include temperature sensors (thermometer), blood pressure monitor, heart (pulse) monitor, blood oxygen monitor, respiration monitor, electrocardiogram (EKG), or any other type of sensor device for generating the sensor data 114 associated with a patient, such as the patient 115.

In this example, the patient 115 is being monitored and the one or more caregiver(s) 119 include a clinician, nurse, medical personnel, lab technician, and/or another caregiver. The system permits interaction between the patient 115 and the caregiver(s) 119 (any type of lab/clinical personnel). In some examples, the caregiver(s) 119 provides laboratory, genomic, and transcriptomic data used in modeling these conditions, including continuous and/or episodic clinical interfacing within the system. For example, episodic data can include data obtained from patient blood draws performed by the clinician over time for updating patient-related health data. The demographic/genomic information may be static, but the labs and other continuous and episodic data is updated dynamically, much like the device ePROs (Electronic Patient Reported Outcomes).

The set of one or more sensor device(s) 106 sends the sensor data 114 to the CRS prediction manager 130 in real-time for utilization in generating CRS predictions and grading of predicted CRS events. The sensor data may be pushed to a computing device or the cloud server 112 from the sensor device(s) 106 via the network 102. In other examples, the CRS prediction manager 130 requests the sensor data from the sensor device(s) 106 and/or the data store 110 storing the sensor data via a pull request. In still other examples, the sensor data 114 is stored until a predetermined time-period or predetermined event triggering transmission of the sensor data to the CRS prediction manager system, such as in a periodic download of the data for use in analysis.

The cloud server 112 is a logical server providing services to one or more connected computing devices or other clients, such as, but not limited to, the patient input device(s) 104, the output device(s) 108 and/or a computing device, such as the computing device 300 in FIG. 3 below. The cloud server 112 is hosted and/or delivered via the network 102.

In some non-limiting examples, the cloud server 112 is associated with one or more physical servers. In other examples, the cloud server 112 is associated with a distributed network of servers.

The cloud server 112, in the example shown in FIG. 1, includes a data store for storing sensor data 114 and/or other patient-related data used by a CRS prediction manager 130 to generate a CRS prediction 118 based on analysis of algorithm input(s) and parameter(s) received from one or more input data sources, such as, but not limited to, the input device(s) 104. The parameter(s) (not shown) optionally include user-configured values and default values, such as, but not limited to, threshold value(s) and/or the predetermined time-period.

The data store 110 is optionally included on the cloud server 112 or accessible by the cloud server 112 for storing data, such as, but not limited to, data associated with a CRS prediction 118 outputs 120 to a user via the output device(s) 108. The outputs 134 include a predicted probability 136 of the CRS event onset and/or predictive grading of any current CRS event or predicted future CRS event which is predicted to occur a pre-determined time-period(s) 122 after prediction generation by the CRS prediction manager system 130 using one or more trained machine learning (ML) model(s) 124. The pre-determined time-period(s) 122 is a user-defined time-period. In this example, the predetermined time-period(s) 122 is a twenty-four-hour time period. However, in other examples, the predetermined time-period(s) 122 is any user-configured time-period during which a predicted CRS event is predicted to occur and/or the predicted CRS event is predicted to change severity (improve or decline).

The data store 110 is optionally implemented as any type of data storage, such as a local or remote data store. The data store 110 can include one or more remote data storage devices accessible via the network 102, such as, but not limited to, the data storage device 312 in FIG. 3 below. In other examples, the data store 110 includes a cloud storage, such as a database or file system on a cloud or a data center. The system can include any configuration of internal and external (networked) storage of the models, parameters, and outputs.

In some examples, the CRS prediction manager system 130 receives sensor data 114 from the sensor device(s) 106 via the network 102 using a communications interface device, such as, but not limited to, the user interface device 308 in FIG. 3 below. In other examples, the CRS prediction manager system 130 requests the sensor data 114 from the set of one or more sensor device(s) 106 via the network 102. The sensor device(s) 106 transmits the sensor data 114 via the network 102 in response to the request. In other examples, the set of sensor device(s) 106 automatically transmits the sensor data 114 to the CRS prediction manager system 130 in real time as the sensor data 114 is dynamically generated. In still other examples, the sensor data 114 is sent at regular intervals or in response to a predetermined event. The sensor data 114 may include continuous sensor data and/or episodic sensor data associated with the vital signs and/or other health data of the patient 115.

As used herein, the term “vital signs” is not limited to patient data input from sensor devices. Vital signs data can also include physiological or biological signals data encompassing a variety of signals and/or features.

The CRS prediction manager system 130, in some examples, utilizes one or more ML model(s) 124. The ML model or models may incorporate any types of techniques to analyze and transform raw sensor data and/or database information to predictive outputs and generate alerts 126. In this example, the ML model(s) 124 are accessed by the CRS prediction manager system 130. In other examples, the ML model(s) 124 can optionally be incorporated within the CRS prediction manager system 130.

The CRS prediction manager system 130, in this example, obtains patient-related health data 109 for a monitored patient 115, including vital signs data obtained from the set of sensor device(s) 106 associated with the monitored patient 115 (patient) and user-provided data associated with the monitored patient 115. The examples are not limited to obtaining patient-related health data from sensors, patients, and/or caregivers. Patient-related data, such as, but not limited to, lab data, EHR data and other data not entered by a user through a specific portal within the system. Patient-related data in other examples is obtained through linked accounts, downloaded from one or more other data sources in addition to or instead of user-provided data.

The CRS prediction manager system 130 analyzes the patient-related health data using a trained ML prediction model selected from the trained ML model(s) 124. The CRS prediction manager system 130 generates the CRS prediction 118 based on analysis of the patient-related health data. The CRS prediction 118 includes a probability of a CRS event occurring within the predetermined time-period(s) 122 after generation of the CRS prediction 118. In some examples, the prediction is output to the user via a notification including the CRS prediction 118, such as a medical or healthcare professional. The notification is output if the probability of the CRS event exceeds one or more threshold(s) 128 indicating onset of the CRS event is likely to occur within the predetermined time-period(s) 122. In some examples, the CRS prediction manager system 130 applies one or more user-configurable threshold value(s) or threshold range(s) or heuristic models to determine if/when to issue a CRS event alert. A threshold range can include a range defined by maximum threshold probability and a minimum threshold probability for generating a notification and/or CRS event alert. If CRS has already onset, the report may or may not additionally include gradation information and deterioration prediction.

In this example, the parameter(s) and/or other algorithm inputs are stored on the data store 110 on the cloud server 112. The data store 110 optionally also stores the prediction(s) grading, and potential for deterioration (predicted outcomes). The threshold(s) are applied to the output probabilities to determine whether to generate one or more alert(s) 126. In these examples, the model(s) are stored and used to transform the patient health data 109 into the CRS prediction.

The data store 110 is optionally used to store patient health data and provides the data to the ML model which is used to monitor the patient and predict CRS events. The data transfer between the data storage and the clinician interface and patient interface are bidirectional, in which the data can be retrieved and also pushed with new data or update the existing data content.

In some examples, the system determines patient similarity measures, such as similarity scores based on the monitored patient's health data such as the patient's history, symptoms and continuously updating vital sign and measurement records to each of the training cohorts, and then uses these scores to select a personalized CRS prediction model from the set of pre-trained CRS predictive models stored in the model(s) 124 in the data store 110. As noted above each of these models are pretrained for a set of similar patients, referred to as the training cohort for the respective model, with similarity measures calculated from their health characteristics including history, comorbid conditions, symptoms, physiological measurements, and laboratory values.

The model(s) 124 in the data store 110, in other examples, also contains a model pretrained for the general population. In the case that the input patient characteristics are a very unique corner cases or found to be less similar to the existing previous patient pool, or if a personalized predictive model does not exist to match for the input patient characteristics, the general population-based model is selected, and the respective model parameters are input to the CRS prediction manager system.

In other examples, the system enables predicting cytokine release syndrome's onset, undertaking associated severity and deterioration monitoring and prediction, generating CRS-related notifications through a CRS prediction manager system, and coupling these system pieces with a patient care pathway to keep patients receiving treatments capable of triggering and susceptible to cytokine storms safe. When a patient is set to receive a treatment that can trigger CRS, information about that patient is gathered. This information includes, but is not limited to, genomic, epigenetic, demographic, diagnosis, disease severity or burden, treatment, prescription drugs, hospitalizations, insurance, health surveys/questionnaires, and laboratory data. This data is then analyzed to stratify the patient's risk profile for an adverse event, including CRS, following the treatment. In triage, the patient is given a certain level of monitoring and proximity to care based on the patient's risk profile. The patient then undergoes treatment and is discharged based on how he/she was triaged. Patient-related health data for a monitored patient is obtained. The patient-related health data comprises vital signs data obtained from a set of sensor devices associated with the monitored patient and user-provided data, including data provided by the patient, any associated patient caregivers, electronic health records, and/or data provided by the clinician associated with the monitored patient. The patient-related data is analyzed using a trained CRS prediction manager system, including one or more trained machine learning prediction models. A CRS event prediction is generated based on the analysis. The prediction includes a probability of a CRS event occurring within a predetermined time-period after generation of the CRS prediction. Multiple predetermined time-periods could be considered at a given time. A notification is provided of the predicted CRS event if the probability of the CRS event exceeds a threshold probability indicating onset of the CRS event is likely to occur within the predetermined time-period. If a CRS event is determined to be onset, the gradation of that CRS event and the probability the patient may further deteriorate within some window of time can also be included in the generated alert, notification, and or report. A monitoring caregiver is able to the report as well as any additional data to make a decision about the need to treat the patient for the adverse event with antipyretics, steroids, vasopressin, oxygen support, etc., and a decision about whether the current level of monitoring is appropriate or not appropriate. In this manner, some examples provide a system by which oncology patients receiving treatment and at-risk for iatrogenic cytokine release syndrome are safely monitored. In an exemplary patient care pathway, the patient is monitored by the CRS prediction manager system 130 following treatment with immunotherapy. If the patient is deemed of be of sufficiently low risk, then the patient is passively monitored with a single wearable device in a remote setting. If the patient is deemed to be of a higher risk, the patient is more actively monitored with attending caregivers and/or with increased number of parameters being gathered. However, the examples are not limited to these care pathways.

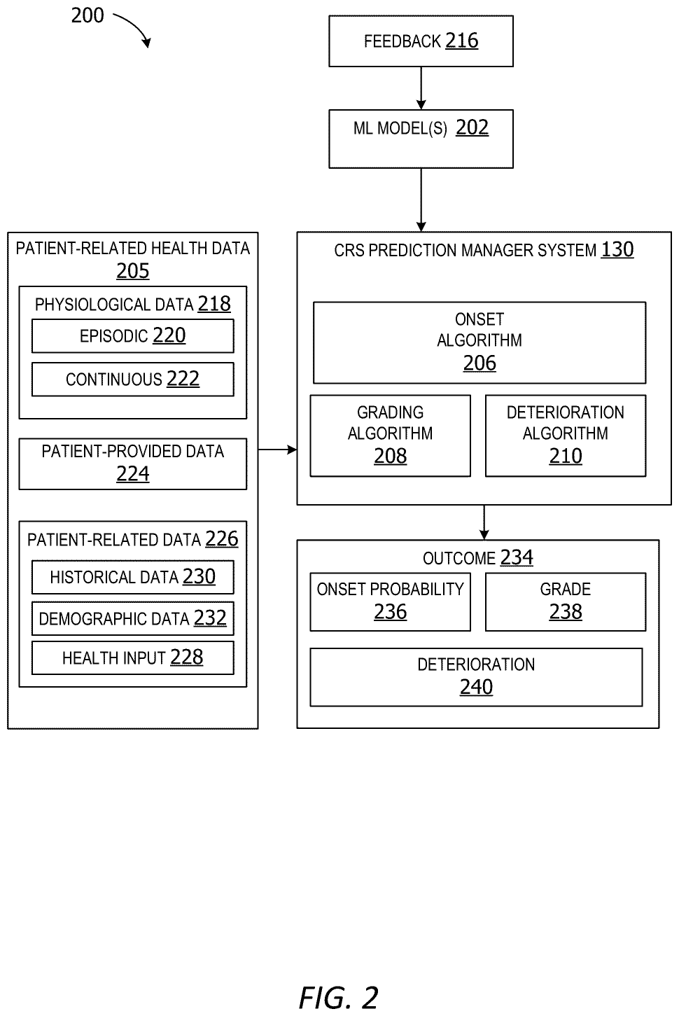

FIG. 2 is an exemplary block diagram illustrating a system 200 for generating CRS event predictions using one or more machine learning (ML) model(s) 202. In some examples, the CRS prediction manager 130 includes a deterioration algorithm 210 which analyzes patient-related health data 205 to predict whether a CRS event is likely to occur in a given patient within a predetermined future time-period. The deterioration algorithm 210, in some examples, determines a probability or likelihood of a CRS event in a patient that is not currently experiencing a CRS event and/or predict whether the condition of a patient already experiencing a CRS event is likely to improve or decline (become worse). Monitoring caregivers in this pathway can use these analyses as an aid in their decision-making when it comes to maintaining or changing patient monitoring conditions as well as deciding how to treat patients who are at-risk for or have adverse events onsetting.

In one sample embodiment, an onset algorithm 206 analyzes patient-related health data 205 to predict when a CRS event is likely to occur following an infusion with an immunotherapy to treat a neoplasm (tumor) in a patient. CRS event onset times vary in length and duration between different immunotherapies. The onset algorithm 206 analyzes patient-related input data to predict whether CRS onset is most likely within a day after treatment, the second day after the treatment, third day after treatment, etc. Treatment can include surgery, immunotherapy, or any other treatment.

A grading algorithm 208 analyzes patient-related health data 205 associated with a user to grade a predicted CRS event in a patient. The ML model(s) 202, in some examples, analyze patient-related health data 205 using pattern recognition to generate the CRS event predictions, onset probabilities and/or grading.

The grade can include a grade selected from a set of two or more grades. For example, the grades can include a first grade for no CRS event predicted and a second grade indicating a CRS event is predicted. The grades can include three grades, such as, no CRS, mild CRS, and severe CRS. The grades can include four grades, such as, no CRS, mild CRS, moderate CRS, and severe CRS.

The grades are not limited to grades of mild, moderate, and severe. In other examples, the grades can include letter grades, percentage grades, number grades, or any other type of grading.

The CRS prediction manager 130, in some examples, utilized trained ML models. One or more of the trained ML models are optionally trained using existing patient data and/or other training data (not shown), such as input data and/or output data to train the algorithm, such as ground truth. The ground truth data, in other examples, includes data reflecting clinical data. In still other examples, the training data optionally includes data associated with historical outcomes.

For example, the training data optionally includes patient vital signs before an immunotherapy infusion as well as through the infusion. The following adverse event(s) can be provided as input training data, while CRS adjudication by professional clinicians following American Society for Transplantation and Cellular Therapy (ASTCT) gradation guidelines can be provided for ground truth output.

The ML model(s) 202 may optionally be updated/retrained dynamically based on updated training data and/or feedback 216 to update/improve the deterioration algorithm 210, onset algorithm 206 and/or grading algorithm 208 used to analyze the patient-related health data 205.

The patient-related health data 205 includes physiological data 218 generated by one or more sensor devices, such as, but not limited to, the sensor device(s) 106 in FIG. 1. In some examples, the patient is intermittently or continuously monitored by one or more home and community based biomedical sensors to generate physiological data 218. The physiological data 218 includes vital signs data.

The sensors may include one or more wearable devices that measure the patient's physical activity, the patient's physiological data, including vital signs, along with other parameters related to patient health. The physical activity measures may include, but are not limited to, body acceleration, steps, posture, fall, activity intensity, ambulation, gait, and associated durations. The physiological measurements may include, but not limited to, heart rate (or pulse rate), heart rate variability, respiratory rate, blood oxygen saturation, temperature, noninvasive blood pressure and correlates, and derived parameters from photoplethysmogram (PPG), electrocardiogram (ECG) or other physiological signals. Additional parameters may include, but are not limited to, those related to edema and swelling such as weight and extremity size. Data may also be collected from non-invasive vital sign sensors provided in a home, community health clinic or general practitioner office (i.e., non-hospital), such as blood pressure monitors and heart rate monitors. Data could also be collected from personal/home invasive/semi-invasive or sample-based sensors such as subcutaneous implantable sensors such as those used for blood glucose monitoring. These can be generally classed as home and community based biomedical sensors (as distinct from hospital-based monitoring equipment). A patient interface is provided to enter the measurement values or connect to and download data from the sensors. This may be directly from the sensors, for example via an app running on a local smartphone or computing device, or from other storage sources, such as cloud storage sources.

A user interface, in other examples, allow collection of patient symptoms. This may be an application installed on the patient's smartphone or tablet (or other computing device) and allows the patient to enter from time-to-time the commonly experienced symptoms associated with his/her health such as cough, fever, pain, nasal congestion, shortness of breath and other signs. This patient health data is sent or uploaded to a patient data store, such as, but not limited to, the data storage device 312 in FIG. 3. This may be secure data storages including, but not limited to, dedicated hard disks on servers or cloud storage services. This may be sent in real time, periodically, or in batches. The monitoring data may be continuous or intermittent, and may comprise repeated measurements of one or more vital signs, with each measurement having an associated time.

The physiological data 218 in other examples, includes episodic 220 data obtained at discrete times/events and/or continuous 222 data obtained from monitors which continuously monitor patient vital signs. In some examples, episodic 220 data includes blood pressure data obtained via a blood pressure device, blood oxygen saturation data obtained via a pulse oximeter, temperature data obtained via a thermometer (contact or non-contact means of thermal sensing). A non-limiting example of continuous 222 data includes but not limited to pulse data generated by a pulse monitor.

Patient-provided data 224 is data provided by a patient, such as answers/response to questionnaires or any other patient-provided information. Other patient-provided data 224 can include symptoms and/or ePROs.

Health input 228 includes any health-related data associated with a monitored patient. Health input 228 in some examples is provided by a caregiver or other medical professional. The health input 228 can include caregiver observations, results of exams, tests performed, notes regarding treatment or any other caregiver provided data. The clinician input can include Glasgow coma scale (GCS) and neurological, cognitive, motor responses, and or AVPU (alertness, verbal, pain, and unresponsiveness) input data. The GCS is a clinician score. Health input 228 can also include genomic data, laboratory test results and other clinician provided input to the CRS prediction algorithm.

Other patient-related data 226 includes data provided by a doctor or other medical provider, historical data 230, patient demographic data 232, or any other patient-related data which may be used by the CRS prediction manager 130 to generate a CRS event outcome 234 for a user. In this example, the patient-related data 226 includes data other than patient-provided data 224 and physiological data 218, such as health input 228, historical data 230 and/or demographic data 232. However, the patient-related data 226 is not limited to health input 228, historical data 230 and/or demographic data 232. The patient-related data 226 can include any type of data associated with a patient obtained from any source associated with a patient, patient health, patient treatment, or other relevant data. For example, it could additionally include genomic, transcriptomic, epigenetic, etc. data.

The CRS event outcome 234, in some examples, include an onset probability 236 that a CRS event will occur during a future time period, such as a twenty-four-hour time period after the prediction is generated. The outcome 234 optionally includes a grade 238 (category) indicating a severity of the predicted CRS event and/or an outcome 234 of an ongoing CRS event. The outcome 234 indicates whether the patient's condition is likely to improve or decline (worsen) within the time period.

In some examples, the onset algorithm 206 identifies the time of event when a CRS condition could onset and output a CRS event versus no CRS event categorical outputs periodically, such as minute, hourly or daily basis. Likewise, the grading algorithm 208 optionally can output different gradation categories or levels if a CRS event is deemed to be onset or about to onset. In still other examples, the grades generated by the grading algorithm 208 a set of CRS severity grades can be mild, moderate, or severe. In yet another example, the set of grades can be grade 1, grade 2, grade 3 or grade 4 (similar to ASTCT grading). In still other examples, the deterioration algorithm 210 generates a deterioration 240 prediction. The deterioration algorithm 210 predicts whether the patient is likely to improve, or decline (deteriorate) based on the output CRS severity grade and/or other available patient-related data. The onset algorithm 206, grading algorithm 208 and/or deterioration algorithm 210 can potentially make use of many ML models for predicting CRS onset at different times, etc. The ML models used by each type of algorithm includes one or more of the ML models from ML model(s) 202.

The patient health data for a patient optionally includes clinical data items obtained from one or more clinical data sources, a plurality of patient measurement data obtained from one or more wearable, home and community based biomedical sensors such as wearable and vital sign sensors, and a plurality of symptoms obtained from the patient, for example via a patient user interface executing on a mobile user device used by the patient, such as, but not limited to, the patient input device(s) 104 in FIG. 1.

The plurality of patients includes historical patients and/or monitored patients. Historical patients are patients for which historical patient health data may be available and includes previously monitored patients. The system may be utilized to monitor a single patient or multiple patients simultaneously.

In some examples, training data and feedback is used to create and/or dynamically update one or more CRS prediction models after the patient has an outcome (resolves). These models include a machine learning (ML) models. The CRS prediction models are stored in a model store, such as a database or file store that electronically stores the relevant model parameters and configuration (for example by exporting a trained model). In some examples, the model store is a data storage, such as, but not limited to, the data store 110 in FIG. 1 and/or the data storage device 312 in FIG. 3. A CRS prediction model is generated, in some examples, by identifying a training cohort of similar patients according to a patient similarity measure and then training the CRS prediction model using the training cohort of similar patients.

In some examples, clinical data input items are divided into demographic/descriptive data of the patient (age, weight, sex, smoking status, etc.), pre-existing medical conditions (diabetes, heart disease, allergies, etc.), and clinical observations/notes. Similarity between patients may be assessed based on correlation measures, scoring systems, distance measures, etc. When generating a specific combination of data items, similarity functions, and/or similarity criterion/criteria used to generate a similarity measure/similar group of patients, a check is performed to ensure the current combination is sufficiently different from another set (for example at least 3 different data items selected). Similarly, after multiple similar patient groups have been identified using different similarity measures, this set could be filtered to exclude a patient group too similar to another patient group to ensure a diversity of similar patient groups, and thus a diversity of CRS models. The models may be trained using all available data for patients (for example using deep learning training methods), or using specific data items, which may be determined based on how the similar patients were identified, for example the same set of data items used to calculate similarity.

A prediction model, in other examples, is generated by training a CRS prediction model on a general population of patients drawn from the plurality of patients. This may be all the patients in the data store or a random or representative sample. Similarity measures could be calculated between patients in the samples to ensure the sample is reflective of a general population (for example by requiring the average similarity to be low). That is, models are trained on a range of homogenous sub-populations with similar health data as well as a model based on a general heterogenous population.

The system may be used to monitor patients to detect or stratify SIRS-related onset events. In the event of CRS onset, the system can grade and predict deterioration in patients. In an example, patient health data is obtained for a monitored patient. The patient health data includes clinical data obtained from one or more clinical data sources, patient measurement data obtained from one or more wearable, home, and community based biomedical sensors such as wearable sensors and/or vital sign sensors (including non-invasive and invasive vital sign sensors), and data describing symptoms obtained from the patient. When the patient is first monitored, patient health data may be captured or imported from electronic health records and clinical record systems, or access may be provided to the electronic health records or systems

The system can continuously monitor the patient collecting regular or ad-hoc patient health data from wearable and home/clinic based vital sign sensors, as well as symptoms. Updates may also be obtained from clinical data sources, such as laboratory test results, treatments, and clinician notes. The system is configured to select a CRS prediction model from the plurality of prediction models for monitoring the patient. This is performed by identifying the prediction model with the training cohort most similar to the monitored patient (i.e., “like patients”), and if no similar training cohort can be identified then selecting the general population prediction model.

The selected prediction model is then used to monitor the patient to detect and/or potentially stratify SIRS-related events, for example by processing new/updates to patient health data. This may be used to generate electronic alerts if an infection and/or CRS event is detected or predicted to happen in future (e.g., within a predetermined time period). Detection is followed by gradation of severity. The system may also repeat the step of selecting the most similar prediction model in response to a change in the patient health data of the monitored patient over time. This allows the system to keep using the most similar (and arguably relevant) patient cohort as the patient's measurements and symptoms change, for example as the monitored patient begins to show signs of CRS.

A clinician user interface is provided, in other examples, to interface with the one or more clinical data sources, such as electronic medical records. The clinician user interface also allows the clinician (including doctors, surgeons, medical specialists and other health care professionals and service providers) to access or visualize the patient's health data and trends from a set of dashboards or summary pages or graphic illustrations on mobile application or website portals, such as via the cloud server 112 in FIG. 1.

In these examples, the clinician inputs clinical interpretations and observational notes into the system via appropriate user interfaces including submitting text summaries of patient status and interactions. Laboratory (lab) test reports, images and documents may also be viewed or imported, uploaded or access granted. The clinician is also optionally able to review push notifications of alerts and/or notifications. The clinician provides feedback regarding whether the generated alerts are true positives or potentially false alarms. The feedback is optionally used to refine/update the ML model.

In other examples, clinician inputs and notes are transferred to the secured cloud/data storage of the system. The clinician interface may also be configured to access electronic medical records stored by hospitals, clinics, or other health providers which contain the patient's demographic characteristic profile (i.e., patient metadata) and clinical history, including outcomes of infections or CRS episodes from previous treatments, any information surrounding inflammation-related biomarkers, previous treatments the patient received, and hospitalizations. Patient metadata, such as demographic information, general health characteristics and pre-existing conditions may also be entered via the patient user interface or clinician user interface. Previous outcomes in relation to CRS events can be used to train the prediction models.

For example, a potential outcome variable predicted by our CRS monitoring solution could be the onset of severe CRS requiring mechanical ventilation. This is an alternative CRS-specific outcome compared to severe CRS requiring use of vasopressor. There may be distinct biomarkers embedded into machine learning-based algorithms for each of these distinct outcomes. For example, previous vascular-related hospitalizations may be indicative of CRS requiring vasopressor. Difficulty dealing with pneumonia or other respiratory infections may indicate pulmonary challenges that are likely to require oxygen support following treatment with an immunotherapy. Certain profiles of inflammation-related biomarkers might be good indicators that the patient is likely to have CRS onset quickly following infusion.

Alternatively, these data can be indicative of the rate at which the CRS worsens. This could include previous outcomes related to infection being associated with the rate at which inflammatory biomarkers in patient serum increase, which could be associated with the CRS gradation. Similarly, previous treatments the patient has received, and their timing, such as fluid resuscitation, can related to the rate at which the patient blood pressure drops, which would be indicative of the rate of increasing CRS severity (patient deterioration). In another example, differences in the changes of inflammatory biomarkers from previous events and outcomes for this specific patient or like patients could be indicative of a more or less severe CRS event or a CRS event as opposed to another SIRS event such as sepsis.

As discussed above, the process to compute the similarity measure could use any or combinations of data items (or encodings) of the patient health data, similarity functions/metrics (which may generate similarity scores), and/or similarity criterion/criteria. There is also no restriction in the methods by which the similarity of various parameters is assessed or how these similarity functions/scores/criterion are combined and or applied to obtain a patient similarity measure. If a matching personalized CRS prediction model exists, the predictive model parameters are input to the CRS prediction manager, which extracts the relevant patient's history, symptoms and vital records from the data storage, and the processed health data trends, laboratory test results, clinician notes from clinician interface tools as well.

The CRS prediction manager system, in other examples, monitors incoming (real-time) patient data. The CRS prediction manager system may be any or a combination of a rule-based CRS event detector, binary or multi-class classifier or a multivariate regressor assessing the risk for CRS events based on the monitoring data. The CRS prediction manager system (ML model) is configured to analyze incoming patient health data and generate an output indicating the risk (probability and grade) of one or more CRS events. This may be a binary outcome, likelihood score or a probability measure. Determination of a positive event or a class or a risk associated with infection and CRS leads to the generation of alerts and notifications. In one embodiment each prediction manager is an ML classifier which is configured to monitor updates to patient health data for the monitored patient and generate an alert if a CRS event is detected.

Alerts may be sent to the patient or their caregiver via the patient user interface, for example to alert them to a potentially serious infection or deterioration event. Alerts may also be sent to the clinician interface to notify the clinician, health care provider and associated parties and displayed in the clinician user interface, for example on a mobile application and or web portal. Additional data such as health trend data may be included with the alert. The clinician can review the generated positive alerts, the corresponding health trend data, and can verify the validity of the generated alerts and provide feedback in annotating the predicted CRS events to be true positives or false positives. In case of a new clinical event, the clinician interface allows the clinician to make entries of clinical events including severe adverse events and changes in medications. The clinician's feedback for the generated CRS events or the new entries of clinical events are pushed and updated as the corresponding reference data for the given patient's health information, measurements, and symptoms.

A decision for retraining of the CRS prediction manager system ML model is obtained either automatically at desired periodic time intervals or with manual confirmation input using clinical interface tool. The automatic decision logic for retraining may be enabled or disabled depending upon desired preset criterion (or criteria). In one embodiment, if the feedback entries for generated positive infection and sepsis events and or the new entries of qualifying clinical adverse events exceeds a preset threshold, then decision logic is enabled for retraining. This results in adaptation or regeneration of the ML models for the given data repository containing patent information, continuous and or discrete patient measurements, and episodic symptoms and reference events. After retraining the models, the updated models are stored in the model store, for example replacing the currently stored models.

FIG. 3 is an exemplary block diagram illustrating a computing device 300 for CRS onset prediction and event grading. In the example of FIG. 3, the computing device 300 represents any device executing computer-executable instructions 302 (e.g., as application programs, operating system functionality, or both) to implement the operations and functionality associated with the computing device 300. The computing device 300 may be any type of computing device implemented as part of a system for CRS onset and severity prediction, such as, but not limited to, the system 100 in FIG. 1 and/or the system 200 in FIG. 2.

The computing device 300, in some examples, includes a mobile computing device or any other portable device. A mobile computing device includes, for example but without limitation, a mobile telephone, laptop, tablet, computing pad, netbook, gaming device, and/or portable media player. The computing device 300 can also include less-portable devices such as servers, desktop personal computers, kiosks, or tabletop devices. Additionally, the computing device 300 can represent a group of processing units or other computing devices.

In some examples, the computing device 300 has at least one processor 304 and a memory 306. The computing device 300, in some examples includes a user interface device 308.

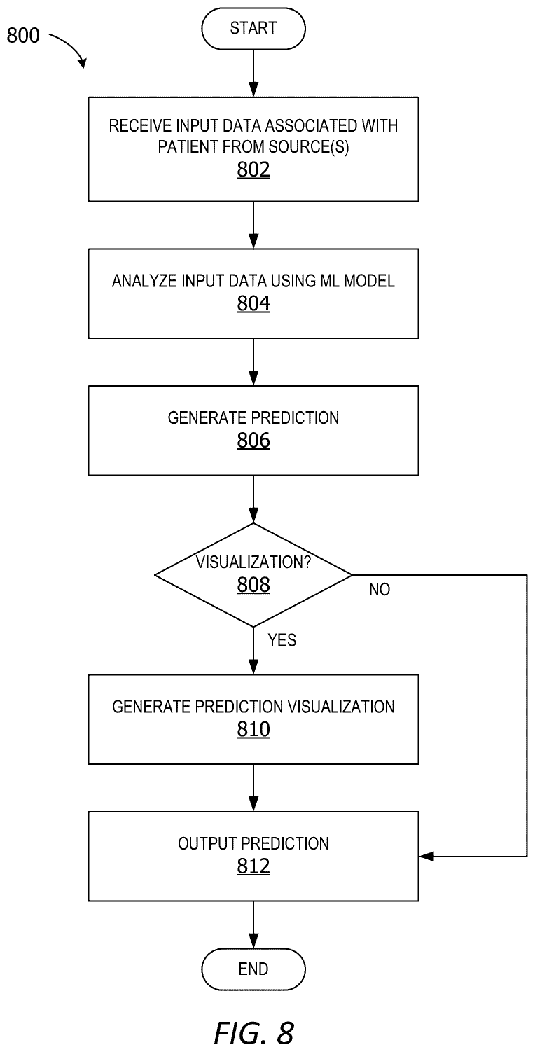

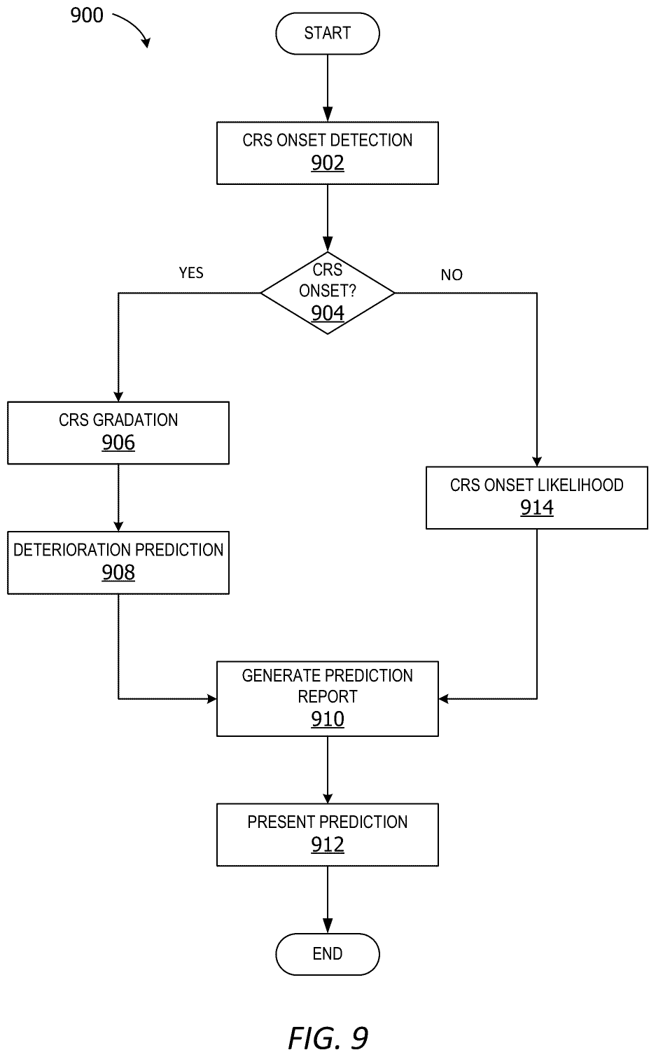

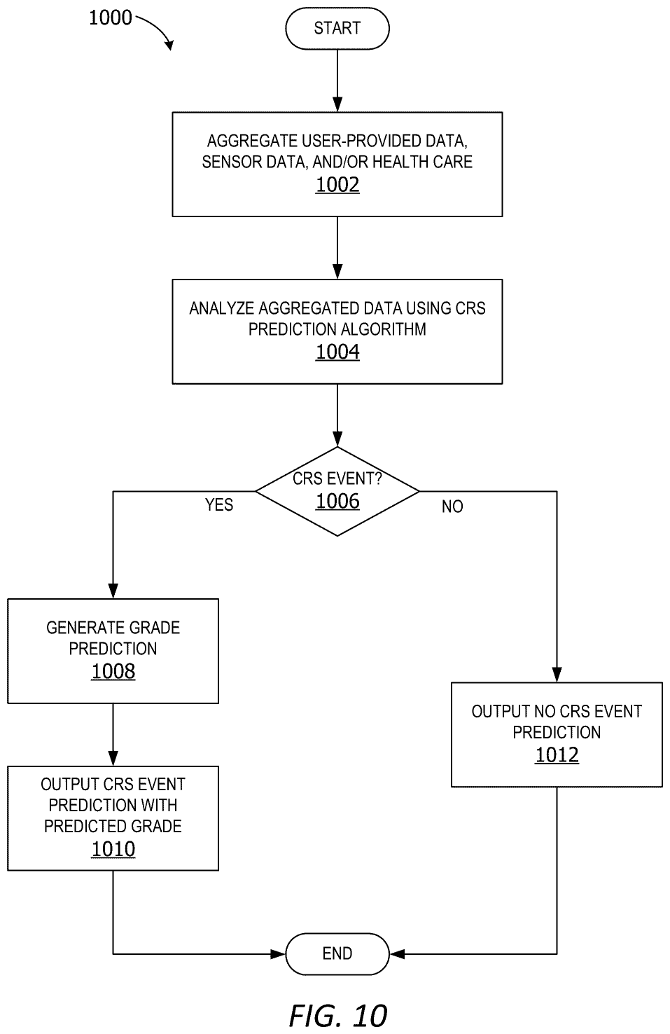

The processor 304 includes any quantity of processing units and is programmed to execute the computer-executable instructions 302. The computer-executable instructions 302 is performed by the processor 304, performed by multiple processors within the computing device 300 or performed by a processor external to the computing device 300. In some examples, the processor 304 is programmed to execute instructions such as those illustrated in the figures (e.g., FIG. 8, FIG. 9, and FIG. 10).

The computing device 300 further has one or more computer-readable media, such as the memory 306. The memory 306 includes any quantity of media associated with or accessible by the computing device 300. The memory 306, in these examples, is internal to the computing device 300 (as shown in FIG. 3). In other examples, the memory 306 is external to the computing device (not shown) or both (not shown).

The memory 306 stores data, such as one or more applications. The applications, when executed by the processor 304, operate to perform functionality on the computing device 300. The applications can communicate with counterpart applications or services, such as web services accessible via a network, such as, but not limited to, the network 102 in FIG. 1 above. In an example, the applications represent downloaded client-side applications that correspond to server-side services executing in a cloud.

In other examples, the user interface device 308 includes a graphics card for displaying data to the user and receiving data from the user. The user interface device 308 can also include computer-executable instructions (e.g., a driver) for operating the graphics card. Further, the user interface device 308 can include a display (e.g., a touch screen display or natural user interface) and/or computer-executable instructions (e.g., a driver) for operating the display. The user interface device 308 can also include one or more of the following to provide data to the user or receive data from the user: speakers, a sound card, a camera, a microphone, a vibration motor, one or more accelerometers, a BLUETOOTH® brand communication module, global positioning system (GPS) hardware, and a photoreceptive light sensor. In a non-limiting example, the user inputs commands or manipulates data by moving the computing device 300 in one or more ways.

In some examples, the computing device 300 optionally includes a communications interface device 310. The communications interface device 310 includes a network interface card and/or computer-executable instructions (e.g., a driver) for operating the network interface card. Communication between the computing device 300 and other devices, such as, but not limited to, a patient input device, sensor device(s) and/or a cloud server, can occur using any protocol or mechanism over any wired or wireless connection. In some examples, the communications interface device 310 is operable with short range communication technologies such as by using near-field communication (NFC) tags.

The computing device 300 optionally includes a data storage device 312 for storing data. The data storage device 312 can include one or more different types of data storage devices, such as, for example, one or more rotating disks drives, one or more solid state drives (SSDs), and/or any other type of data storage device. The data storage device 312 in some non-limiting examples includes a redundant array of independent disks (RAID) array. In other examples, the data storage device 312 includes a database.

The data storage device 312, in this example, is included within the computing device 300, attached to the computing device 300, plugged into the computing device 300, or otherwise associated with the computing device 300. In other examples, the data storage device 312 includes a remote data storage accessed by the computing device 300 via a network, such as a remote data storage device, a data storage in a remote data center, or a cloud storage.

In this example, the data storage device 312 optionally stores inputs 314 utilized by the CRS prediction manager system 130 to generate a CRS prediction 316. The CRS prediction 316 outputs 318 in some examples includes a probability 320 indicating a likelihood of CRS onset and a grading 322 indicating a degree of severity of the CRS onset and/or predicted outcome if CRS should occur. The outputs 318 are provided to a user via the user interface device 308. In some examples, an alert generation 324 generates an alert indicating the prediction 316, such as, but not limited to, the alert lxx in FIG. 1 above.

In other examples, the outputs 318 are provided to the user via one or more notification(s) 326 provided to the user via the user interface device 308. In this example, the notification(s) 326 are displayed or otherwise output via the user interface device on the computing device 300. In other examples, the notification(s) 326 are transmitted to a remote device for viewing and/or presentation to the user via an output device, such as, but not limited to, the output device(s) 108 in FIG. 1 above.

The CRS prediction manager system 130, in some examples, generates a prediction visualization 328 associated with the prediction 316. The visualization 328 is optionally the prediction 316 and/or other patient-related data associated with the prediction 316 presented in a visualization, such as a graph, table, chart, report, or other visual. The visualization can also include explain-ability metrics for the prediction/output.

The notification(s) 326 include one or more message(s) and/or alert(s) associated with the prediction 316. The notification(s) 328n may be output to a diagnostician, medical provider, or any other user via the user interface device 308 or other I/O device. In other examples, a notification may be transmitted to a remote computing device or server via a network, such as the network 102 in FIG. 1.

Thus, in some examples, the notification(s) 326, including the prediction 316, is output to one or more users, such as a medical or healthcare professional if the probability of the CRS event exceeds a threshold probability indicating onset of the CRS event is likely to occur within the predetermined time-period. The alert generation 324 component includes or applies one or more user-configurable threshold value(s) or threshold range(s) or heuristic models to determine if/when to issue a CRS event alert. A threshold range can include a range defined by maximum threshold probability and a minimum threshold probability for generating a notification and/or CRS event alert. If CRS has already onset, the report may or may not additionally include gradation information and deterioration prediction.

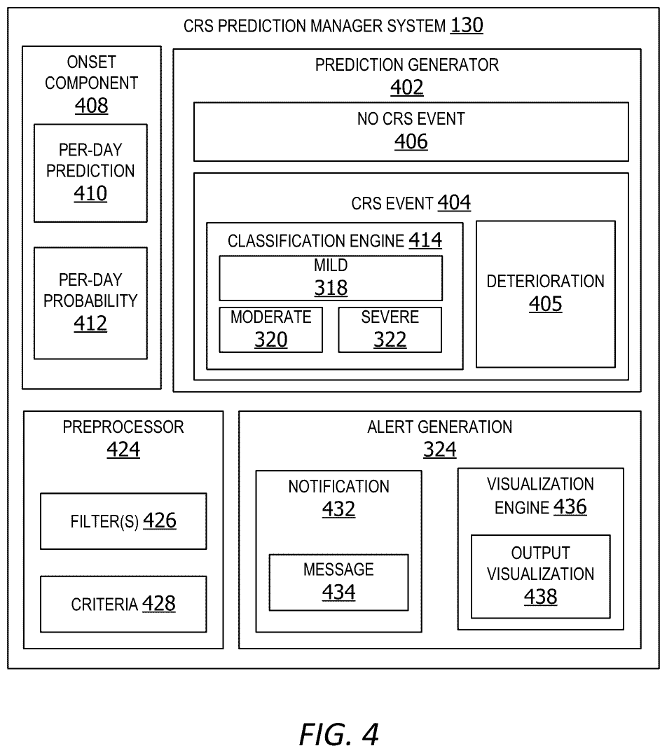

FIG. 4 is an exemplary block diagram illustrating a system 200 including CRS prediction manager system 130 for generating CRS event alerts relating to probability of onset or deterioration and the grade of severity. In some examples, the CRS prediction manager system 130 includes a prediction generator 402. The prediction generator 402 analyzes sensor data using a prediction algorithm to determine whether a CRS event 404 is likely to occur. The prediction generator 402 outputs an indicator that no CRS event 406 is likely to occur if the sensor data analysis does not indicate a CRS event is probable. In other words, the system can optionally separate sepsis and other SIRS conditions, the “no CRS event” prediction can optionally incorporate SIRS conditions and indicate CRS is not emerging or it could indicate that an adverse event is expected that is not CRS. The system could optionally also output an indication/prediction that no adverse event (no CRS or any other adverse event) is predicted.

In other examples, the CRS prediction manager 130 includes an onset component 408 analyzing patient-related data, including the sensor data, to determine whether CRS event onset is likely on a given day or within a given time-period. In this example, the onset component 408 generates a per-day prediction 410 indicating whether CRS onset is likely on a given day following a medical procedure, such as surgical intervention. The onset component 408 optionally also generates a per-day probability 412 indicating a predicted probability of CRS onset on a given day or within a given time-period.

If a CRS event is predicted to occur in future or if a CRS onset is currently occurring, the CRS prediction manager system 130, in some examples, generates a deterioration 405 prediction. The deterioration 405 prediction indicates a probable outcome of the current CRS event. The deterioration 405 prediction can indicate whether a patient is likely to improve within a given period of time or indicate whether the patient's condition is likely to deteriorate within a given future time period based on the patient's current condition. This deterioration prediction is generated using a deterioration algorithm, such as, but not limited to, the deterioration algorithm 210 in FIG. 2.

If CRS onset has occurred or is predicted to be likely to occur, a classification engine 414 generates a grade for each predicted CRS event. The grade indicates the predicted severity of a CRS event. The set of grades can include two or more possible grades. The grades in this example include a grade of mild 418, moderate 420 and severe 422. In this example, the set of grades includes three grades. In other examples, the set of grades includes two grades, four grades, five grades or any other number of grades for grading the severity of a predicted case of CRS for a given patient. Thus, the system decouples deterioration and grading to predict either deterioration or grading, as well as predicting both deterioration and grading. In an example, if the system determines CRS of a certain grade has onset, the system proceeds to determine if the CRS is getting worse (deterioration). Likewise, if the system predicts no CRS event, a determination of grading or deterioration prediction is unnecessary.

The CRS prediction manager system 130 optionally includes a preprocessor 424 that filter(s) 426 input data using one or more user-configurable criteria 428. The preprocessor filters the input data prior to processing of the input data for prediction generation.

An alert generation optionally generates a notification output to the user to notify the user as to the predicted onset, grading and/or outcome of the predicted CRS event, if any. The notification can include a message 434, such as an alert, instructions, suggested actions (treatments), etc.

In still other examples, the CRS prediction manager system 130 includes a visualization engine 436 which generates an output visualization 438 presenting the generated prediction in a visualization for user viewing. The visualization can include a graph, chart, table, graphic, color coding, or any other type of visualization. Such visualizations allow for monitoring caregivers to make rapid assessments about the trajectory and/or current state of the patient. This aids in decision making regarding whether the patient's monitoring should be changed or if treatment or change of treatment for the patient is necessary.

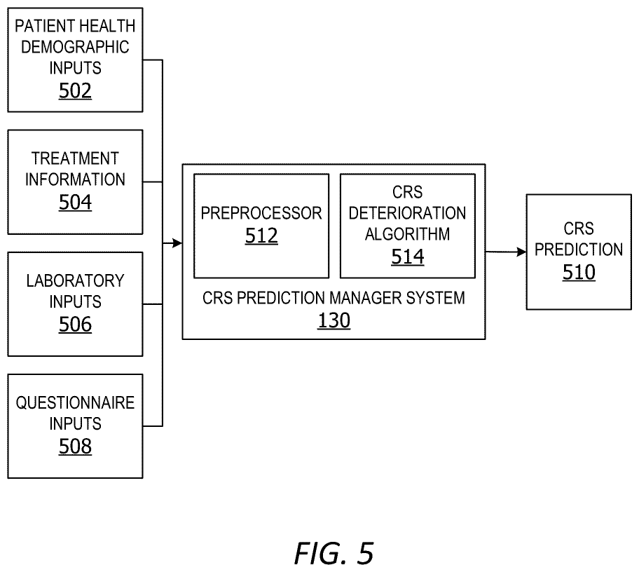

FIG. 5 is an exemplary block diagram illustrating a CRS prediction manager system 130 including a CRS deterioration algorithm for predicting and alerting patients and caregivers regarding a patient's likelihood to deteriorate following CRS onset. The CRS prediction manager system 130 receives input data, including patient health/demographic inputs 502, treatment information 504, laboratory inputs 506 and/or questionnaire inputs 508 provided by the patient in response to questions on one or more questionnaires. The input data is filtered by a preprocessor 512. In some examples, the preprocessor 512 is a pre-processor component, such as, but not limited to, the preprocessor 424 in FIG. 4.

The CRS deterioration algorithm 514 analyzes the input data and generates a CRS deterioration prediction 510. In this example, the CRS prediction manager system 130 includes a grading of CRS and prediction of deterioration where CRS has onset. The deterioration prediction can include analysis using a CRS deterioration algorithm, such as, but not limited to, the deterioration algorithm 210 in FIG. 2.

The prediction 510 indicates whether a CRS event is predicted to occur and/or whether the patient's condition is predicted to deteriorate within a given time-period after the prediction is generated or whether no CRS event is predicted. The time-period following the prediction generation is a user-configured time-period. In this example, the prediction indicates whether a CRS event is probable within twenty-four hours the prediction is generated.

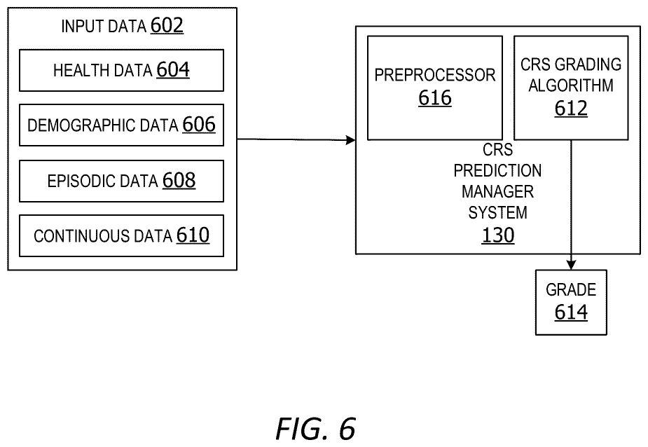

FIG. 6 is an exemplary block diagram illustrating a CRS prediction manager system 130 including a CRS grading algorithm 612 for generating a predicted grade 614 associated with a CRS onset. The CRS prediction manager 130 receives input data 602, such as, but not limited to, health data 604 associated with a patient, demographic data 606 for the patient, episodic data 608 generated by one or more sensor devices, and/or continuous data 610 generated by one or more sensor devices, such as, but not limited to, the sensor device(s) 106 in FIG. 1. In some examples, the health data 604 is data associated with a patient, such as, but not limited to, the health data 109 in FIG. 1.

A preprocessor 616 optionally applies a filter to filter out unwanted or inapplicable data. The filtered data is analyzed by the CRS grading algorithm 612 to assign a grade 614 to a current CRS event or a predicted future CRS event. The grade indicates the severity of the current or predicted future CRS event.

The input data 602 is not limited to the input data shown in FIG. 6. In other examples, input data utilized by the prediction manager can include genomic data, transcriptomic data, laboratory data, diagnostic test results, patient-provided responses to questions, caregiver observations, as well as any other type of input data associated with a monitored patient's health.

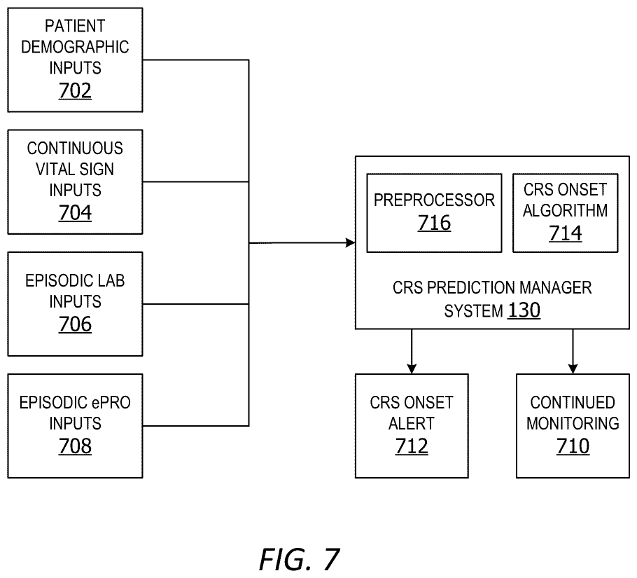

FIG. 7 is an exemplary block diagram illustrating a CRS prediction manager system 130 including a CRS onset algorithm 714 for predicting CRS onset and generating a CRS onset alert 712. The input data, in this example, includes patient demographic inputs 702, continuous vital sign inputs 704, episodic lab inputs 706 and/or episodic ePRO inputs 708. Episodic ePRO inputs 708 includes user responses to questions provided to the patient via an electronic device, such as the input device(s) 104 in FIG. 1 and/or the computing device 300 in FIG. 3.

In some examples, the CRS onset algorithm 714 analyzes filtered data generated by the preprocessor 716 to determine whether CRS onset is probable within a predetermined future time-period. The preprocessor 716, in some examples, is a preprocessor such as, but not limited to, the preprocessor 424 in FIG. 4.

If no CRS event onset is probable, the system continues monitoring 710. If a CRS event is likely, the CRS prediction manager 130 outputs a CRS onset alert 712 to one or more users. The CRS onset alert 712 is optionally output via a notification and/or a visualization. The CRS onset alert 712 may be provided to the user via a user interface device. In other examples, the CRS onset alert is transmitted to a remote computing device via a network.

Thus, if CRS has onset, the CRS is graded, and potential deterioration is predicted. If CRS onset has not occurred, the probability of onset is assessed, as shown in the exemplary process flow of FIG. 6.

FIG. 8 is an exemplary flowchart 800 illustrating a processor for generating a CRS event prediction visualization. The process shown in FIG. 8 is performed by a CRS prediction manager component, executing on a computing device, such as the computing device 300 or the patient input device(s) 104 in FIG. 1.

The process begins by receiving input data associated with a patient from one or more source(s) at 802. The source(s) can include sensor device(s), a data storage device, a cloud storage, a patient file, clinician provided data, patient provided data, etc. The input data is analyzed by a CRS prediction manager using a ML model at 804. A CRS prediction is generated based on the analysis at 806. A determination is made whether to generate a visualization of the prediction at 808. If yes, a prediction visualization is generated at 810. The visualization can include charts, graphs, or any other type of visual representation of the prediction. The prediction is output at 812. The process terminates thereafter.

While the operations illustrated in FIG. 8 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 8.

FIG. 9 is an exemplary flowchart 900 illustrating a process flow for generating a CRS prediction. The process shown in FIG. 9 is performed by a CRS prediction manager system component, executing on a computing device, such as the computing device 300 in FIG. 3 or the patient input device(s) 104 in FIG. 1.

The process begins with CRS onset detection at 902. In some examples, a caregiver performs an assessment to determine if the patient is currently experiencing CRS at 904. If the patient is experiencing CRS (CRS onset) at 904, CRS gradation 906 is performed to determine the grade. A deterioration prediction is made at 908 based on the CRS grade. A prediction report is generated at 910. The prediction is presented to one or more users at 912. In some examples, the report is presented to a user, such as a caregiver or other medical personnel via a user-facing interface or other output device.

Returning to 904, if CRS onset has not occurred, such as where the patient is not currently experiencing CRS, a likelihood of CRS onset likelihood 914 is calculated. In other words, if the patient is not experiencing CRS, the system determines the chance of CRS onset (or CRS onset of a certain grade) and generates a prediction indicating the likelihood of onset. The prediction report is generated at 910 and presented to the user at 912 via an interface or other output device. The prediction report may be presented to the user via an interface or output device associated with the computing device generating the prediction or the prediction report may be transmitted to a remote user-facing output device via a network connection, such as, but not limited to, the network 102 in FIG. 1. In some examples, the CRS standard for what is CRS (CRS onset) is CRS grade greater than or equal to grade two (CRS grade >=2) for example. The process terminates thereafter.

While the operations illustrated in FIG. 9 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 9.

FIG. 10 is an exemplary flowchart 1000 illustrating a process for generating CRS event prediction with a predicted grade. The process shown in FIG. 10 is performed by a CRS prediction manager component, executing on a computing device, such as the computing device 300 in FIG. 3 or the patient input device(s) 104 in FIG. 1.

The process begins by aggregating user-provided data, sensor data, and/or health data at 1002. The aggregated data is analyzed using a CRS prediction algorithm at 1004. A determination is made whether a CRS event is likely at 1006. If yes, a grade for the predicted CRS event is generated at 1008. The CRS event and grade prediction output is 1010. If no CRS event is predicted at 1006, a no CRS event prediction is output at 1012. The process terminates thereafter.

While the operations illustrated in FIG. 10 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 10.

In some non-limiting examples, training the predictive ML model training begins by retrieving an extensive patient dataset consisting of electronic health records (EHRs) from tens of thousands of intensive care unit (ICU) patient stays. These records optionally include patients' demographic information, discharge diagnoses, events during the patients' stays such as manually recorded vitals, labs, and treatments. To begin CRS model development, a cohort of oncology patients is extracted from the patient dataset that did not have indications of infection nor sepsis to explain an immune response. The designation of a patient as an oncology patient without infection or sepsis was made via ICD-9 codes. Patients were excluded if they had an infection indicated by having an ICD-9 code for an infectious disorder. Sepsis patients were also excluded.

For patients with multiple stays in the dataset, in one example, data associated with only the first stay was included in the dataset. Additionally, patients who were under the age of 18 and patients who died during their admission were excluded. After filtering for these inclusion and exclusion criteria, n=1,139 patients with 9,892 days of patient data were extracted. Close examination of a subset of patient medical records revealed that a lot of patients were receiving surgical interventions and standard of care treatments such as chemotherapy or radiation therapy for their cancers. These surgical, pharmacological, or other clinical interventions can result in immune responses, hypoxia and hypotension stemming from a host of issues, including infection, which can be hard to disentangle or conclusively rule out. The shortcomings of this population are noted. This serves as one extended example of extracting a cohort of patients susceptible to CRS in a publicly available dataset and developing machine learning based models to monitor and grade these patients for CRS of certain seventies. It is not limiting the types of patients that could be extracted and monitored, the datasets that could be generated, or the types of machine learning-based models that could be developed. Patients receiving immunotherapies could be targeted, etc.

In a continuation of the example given, subsequent to the patient extraction, the individual patient days in the ICU were labelled. The goal of the CRS prediction grading algorithm (classifier) was to predict the CRS grade of a patient during the following twenty-four-hours. The mildest CRS grades, in these examples, are defined by fever and managed hypoxia and hypotension; the most severe grades, in these examples, require life-saving intervention, e.g., mechanical ventilation (hypoxia) or vasopressors (hypotension).

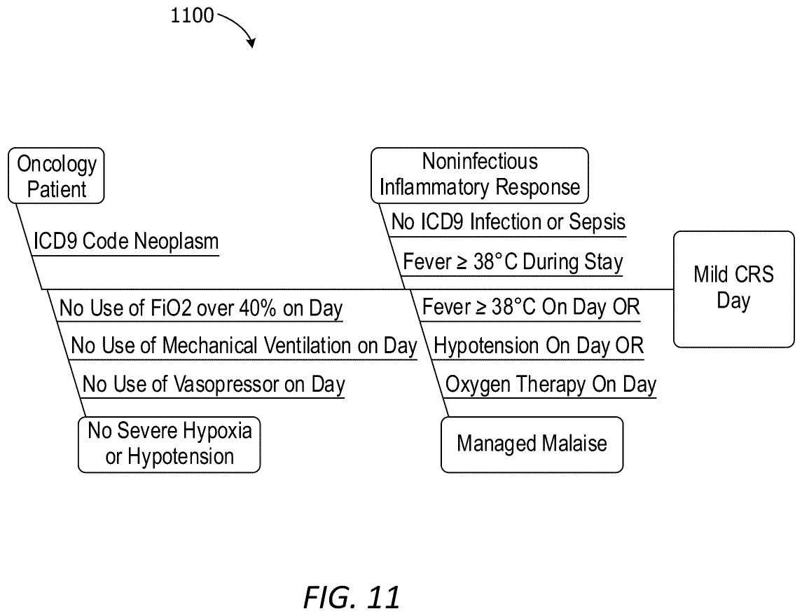

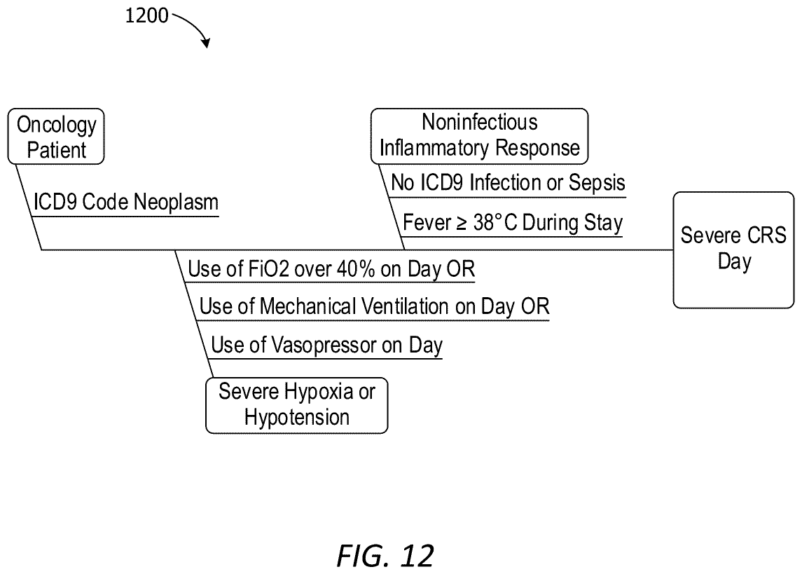

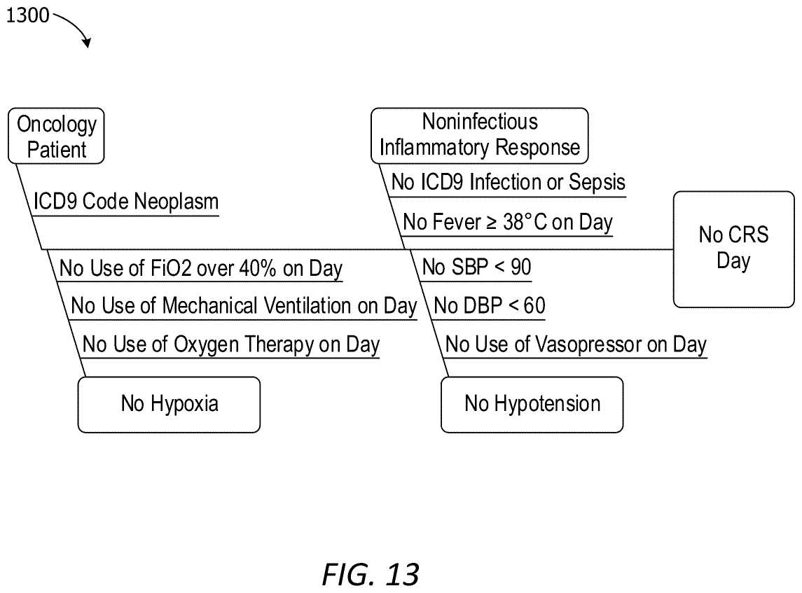

In this specific example, which is not limiting, the time of a patient's admission is considered as the initial time. Each 24-hour period following the initial time of admission until the patient is discharged for which there was data in the EHR was labelled with an outcome variable. If a patient was treated for a severe condition, with a vasopressor, mechanical ventilation, or fraction of inspired oxygen (FiO₂) greater than 40%, then the day was labelled as a severe CRS day. If the patient had mild symptoms, fever and hypotension, or less serious treatments, such as oxygen supplement with inspired oxygen at less than 40%, but did not receive the more serious treatments, then the day was labelled as mild CRS day. If the patient had none of the treatments or symptoms indicated, then the day is labelled as a no CRS day. This process is summarized in FIG. 11, FIG. 12, and FIG. 13 below.

Referring now to FIG. 11, an exemplary summary 1100 of ground truth rules used to extract and label a patient day during which the patient is experiencing CRS of mild severity. FIG. 12 is an exemplary summary 1200 of ground truth rules used to extract and label a patient day during which the patient is experiencing severe CRS. FIG. 13 is an exemplary summary 1300 of ground truth rules used to extract and label a patient day during which the patient is experiencing no CRS.

The examples shown in FIG. 11, FIG. 12 and FIG. 13 provides exemplary guideline definitions of CRS grades for ICU patient days. The example sets of rules are not meant to provide an encompassing or limiting definition of the condition. The rules for predicting CRS onset, labeling severity or outcomes may vary with the various types and examples of the CRS identification and grading system.

The definition of CRS can vary across studies, treatments, clinics, and as additional information about the pathophysiology of the condition is learned. CRS, sepsis, and neurotoxicity are differing conditions. The guideline definition of CRS in each context determines true positives and false positives when considering prediction of onset, grading, and deterioration prediction. The definitions in different contexts indicate what should be identified and graded or ruled out as separate SIRS conditions such as sepsis, neurotoxicity, etc. After checking for these conditions, the 9,892 extracted patients days consisted of 5,215 no CRS days, 1,931 mild CRS days, and 2,746 severe CRS days.

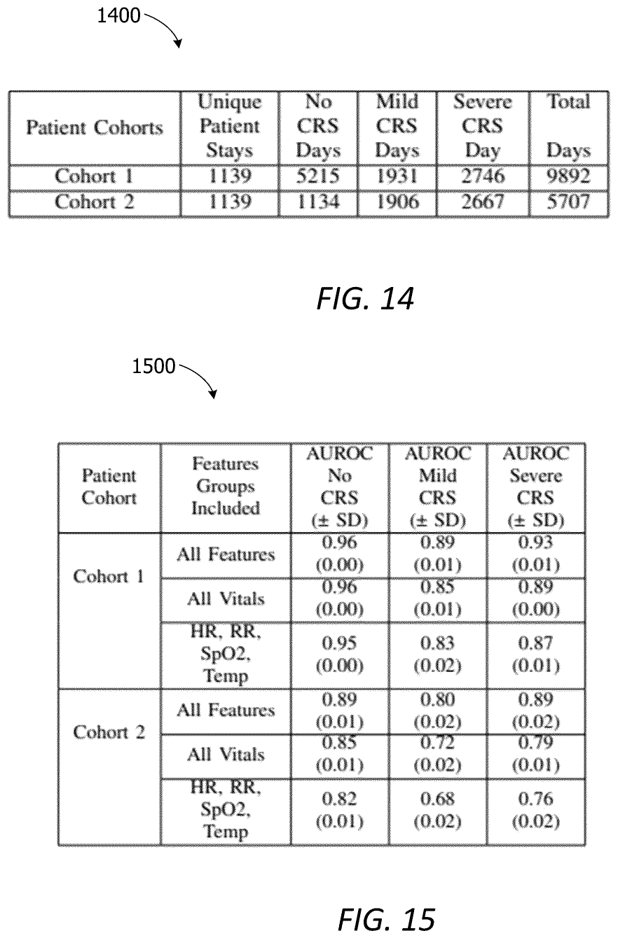

Referring now to FIG. 14, an exemplary table 1400 illustrating a summary of unique patients' days and CRS severity conditions identified and or extracted from a dataset. In this example, there were 9892 patient days extracted via the procedure described above. The majority of these days were demarcated as no CRS days (5215 days). A manual examination of the data revealed almost 80% of the no CRS days had no patient monitoring data in the preceding twenty-four-hours compared to approximately 2% of mild CRS days and approximately 3% of the severe CRS days. A second cohort of patient days required vitals and/or GCS data within the twenty-four-hours before the prediction. The second cohort consisted of the same 1,139 patients but with 1,134 no CRS days, 1,906 mild CRS days, and 2,667 severe CRS days.

The input, in these examples, fall into two general groups, vitals and GCS features. The vitals consisted of heart rate, respiration rate, body temperature, SpO2 levels, systolic and diastolic blood pressure. The GCS features are the verbal, motor, and eye-opening response scores. The features derived from these measurements include the extreme values from the data in the last twenty-four-hours as well as all days preceding the last twenty-four-hours. These were the only features used in the set of models for the first cohort. The models for the cohort requiring data within twenty-four-hours of prediction contained additional statistical moments and trends from these data. In both groups, various combinations of features were evaluated to determine the resulting performance when only a subset of the vitals or GCS data was used.

The output of the models is the probability of the patient having no CRS, mild CRS, or severe CRS during the twenty-four-hours following the prediction time using all of the patient's data up until the time of prediction. The probabilities sum to one. The models in this example are XGBoost models. Any machine learning model-type could have been chosen for this example, and the use of XGBoost should not be limiting in any way.

The basis of XGBoost models are decision trees. Decision trees split training data into subgroups with single classes being over-represented based on informative feature values. For a given decision tree, a test point follows a path along the tree based on its features' values and the learned splits to a leaf node; the probability of that test sample belonging to a certain class depends on the proportion of examples of that class at that leaf node. XGBoost devises decision tree models using the training data. Each decision tree gets training examples wrong. Additional trees can be added that accurately predict examples previously missing. Trees can be added and weighted depending upon their performances on the training examples. The prediction for a test point is the weighted sum of the predictions of the trees.

In one example model evaluation, six ML models are developed and tested with two sets of three models each. However, the examples are not limited to six ML models. In other examples, two ML models, as well as three or more ML models can be used. The examples can make use of potentially any number of ML models to CRS onset at different times, etc.

In this example including six ML models, the first set of three models are the models that did not require vitals or GCS data within the twenty-four-hours leading up to the time of prediction. The second set of three models utilized vitals and/or GCS data within the twenty-four-hours leading up to the time of prediction. Each of the sets had one model that incorporated features from all nine data types discussed above (vitals and GCS), a second model that incorporated only the vital sign data types discussed above, and a final model that incorporated only heart rate, respiration rate, SpO2 and body temperature features. These models, in this example, became increasingly more suited for remote and continuous patient monitoring. The data types are accurately measurable with common wearable devices. Five-fold cross validation is used in this example to validate each of the six models. Days are split randomly amongst the five folds while balancing no CRS, mild CRS, and severe CRS grades.

The splits, in the example shown in FIG. 14, are not even because patient stays were contained to a single fold to ensure there was not any data leakage. A single patient's day data was not incorporated for both training and testing. Performance metrics were calculated as the mean standard deviation of the area under the receiver operating characteristic curve (AUROC).

Turning now to FIG. 15, an exemplary table 1500 illustrating AUROC statistics for various models incorporating various input feature sets and separating CRS grades or gradations is shown. The results for the six models are summarized in the exemplary table 1500 illustrating AUROC statistics for various models involving various choices of input feature sets and separating CRS grades or gradations. As shown in the table of FIG. 15, there is evidence that the no CRS event, mild CRS event, and severe CRS classes are well separated regardless of the data used when using the patient day cohort that does not require vitals or GCS data within the 24 hrs before the time of prediction. The models separating severe CRS from non-severe CRS (no CRS and mild CRS) had a minimum AUROC of 0.87 and separating no CRS from CRS (mild or severe) had a minimum AUROC of 0.95. The low standard deviations in performance across the five folds for all models shows the consistency with which these classes are separable.

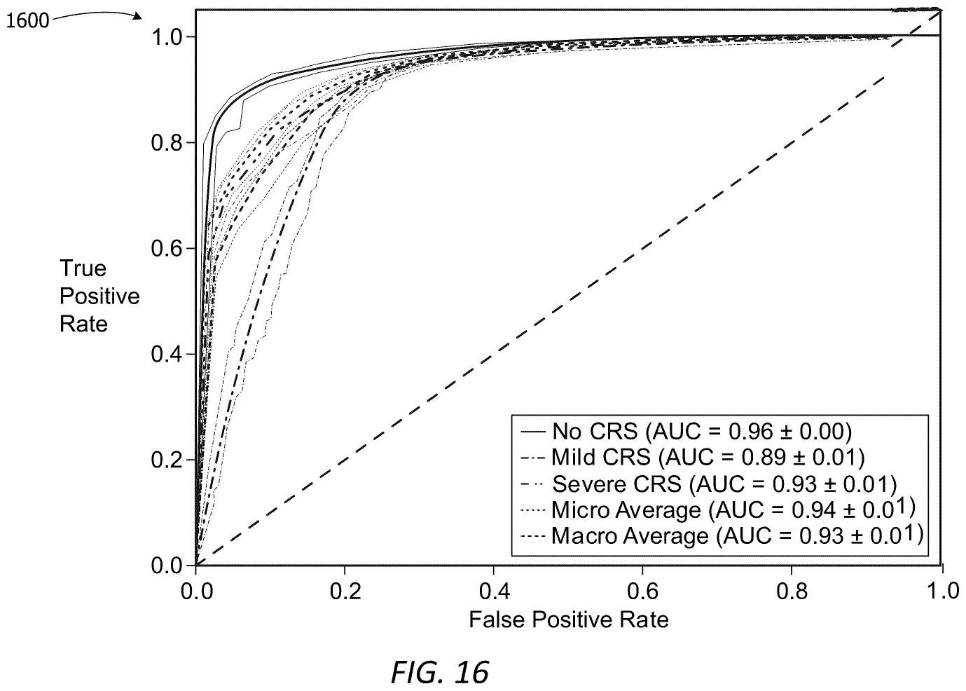

FIG. 16 is an exemplary graph 1600 illustrating all feature ROC curves for an exemplary first patient cohort. The ROC for the model using all features is shown in FIG. 16. In some examples, GCS scores and BP data are predictive of the CRS grades of patients in the following twenty-four-hours. There are statistically significant (p<0.05) differences between the average AUROCs when using all features, when removing GCS, and when removing GCS and BP features for models separating severe CRS from non-severe CRS, mild CRS from the other two conditions, and no CRS from CRS. If assumptions of normality held, using a Repeated Analysis of Variance (ANOVA) with paired post hoc t tests if significant difference is found in the ANOVA. If normality did not hold, a Friedman test with Nemenyi post hoc testing is used.

In all three model types, there is a significant difference between groups. All group pairs were significantly different when separating severe CRS from non-severe CRS and no CRS from CRS. To address the issue of missing data being predictive, consider the models that predicted CRS grades on days for which there was vitals and/or GCS data in the preceding twenty-four-hours. These models are intended to be more practical as they attempt to not exploit the different monitoring levels. These models show predictive value in discriminating the three classes in the twenty-four-hours following prediction with a minimum AUROC of 0.68. An AUROC of 0.76 separates severe CRS and non-severe CRS using only 4 vital signs. AUROC statistics are highest for no CRS vs. CRS.

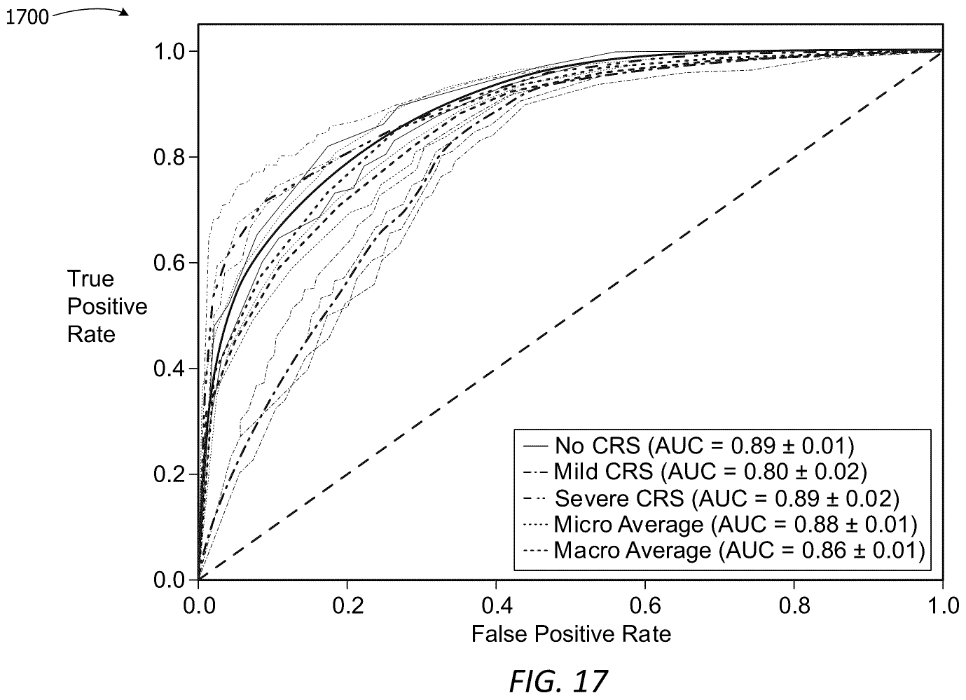

FIG. 17 is an exemplary graph 1700 illustrating all feature ROC curves for an exemplary second cohort. ROCs for the all-features model are shown in FIG. 17. Here, performance is consistent across the five folds.

With these three model types, statistical differences between the performances of models incorporating different feature groups are assessed using the same procedure as above. There is a statistically significant difference (p<0.05) between all pairings of groups for all model types. The all-feature model is the highest performing. The model using all vital signs performed better than the model using HR, RR, SpO2 and body temperature. The importance of GCS and BP is supported by looking at the average feature importance from the five-fold cross validation for the all-feature model and the vitals-only model.

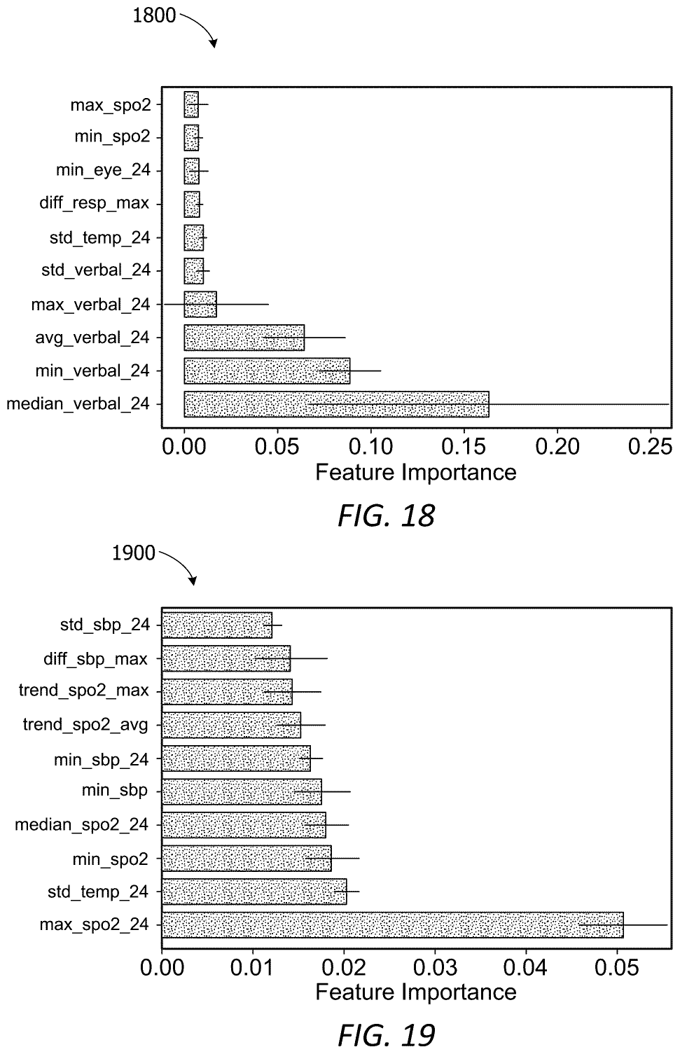

FIG. 18 is an exemplary XGBoost feature importance graph 1800 illustrating the most predictive features for grading CRS in one example patient cohort when numerous feature types were incorporated as input. FIG. 19 is an exemplary XGBoost feature importance graph illustrating the most predictive features for grading CRS in one example patient cohort when only vital signs were incorporated as input. The top ten features are shown in the graph 1800 in FIG. 18 and the graph 1900 in FIG. 19 below. The various data sources are important predictors of CRS grades in the following twenty-four-hours.

In another non-limiting example, the CRS prediction manager includes predictive models that allow for the continuous and remote monitoring of patients at risk for CRS and assessing CRS severity on a more dynamic daily basis with promising accuracy and precision. CRS is a noninfectious SIRS condition that is common in oncology, and, if it progresses to severe grades, it is dangerous and costly for the affected patient. A cohort of oncology patients who could be at risk for CRS is extracted from the dataset. These patients do not have ICD-9 codes indicating treatment for infection or sepsis, but many of them developed fever along with signs of hypotension and/or hypoxia. These patients' days are labelled with CRS grades following the latest guidelines. The extracted data is analyzed to understand the shortcomings of existing data for understanding and predicting this condition.

Models are constructed to predict the patients' CRS grade in the following twenty-four hours. The different models incorporated data from different types of environments to allow for predictions to be done most accurately depending on where a patient might be monitored.

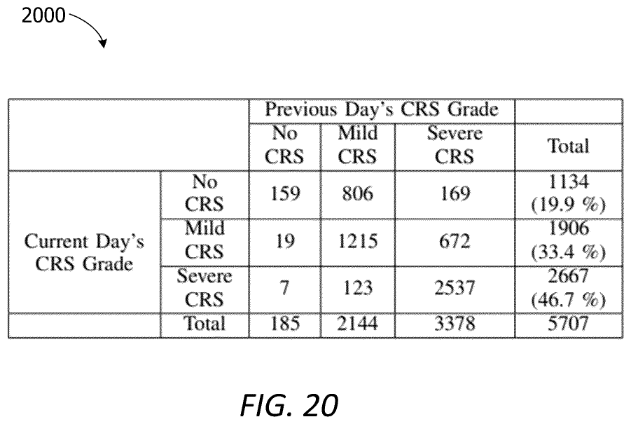

FIG. 20 is an exemplary table illustrating transition matrix data for daily patient CRS grades showing patient CRS grades from a current day to the next. As shown in FIG. 20, the data extracted from the dataset had patients, in general, staying at the same CRS grade day-to-day or getting better. Over 86% of no CRS patient days stayed the same one day to the next day (159/185). Patients with mild CRS often improved (806/2144) or stayed the same (1215/2144). Severe CRS grade days stayed the same into the next day 75.1% (2537/3378) of the time. This is because of the type of dataset from which this exemplary cohort was extracted from. Other exemplary cohorts, such as those used when predicting CRS onset, would have different patient journeys. This is just an example of one analysis that would be done for a specific cohort and specific CRS labeling procedure. Patients receiving immunotherapy, for example, could have a more parabolic-shaped patient journey, deteriorating in the time following infusion before ameliorating in response to steroid-based immunosuppressive treatments.

Closer examination of the patients in the current example's EHRs showed that many of these patients were receiving surgical interventions for their cancers. Patients received treatment throughout the entirety of the data gathering and were closely monitored by clinical professionals. These factors are specific to this example and are meant to show the range of patient types that can be extracted under different definitions of CRS and the range of monitoring environments in which CRS monitoring and predictive solutions can be deployed. Other examples focus on patients receiving immunotherapy treatments.

This specific example has shown how the cohort extracted vital sign-based features to allow for remote patient monitoring of CRS. The models developed in this work showed high predictive value for CRS grades for twenty-four-hours following the time of prediction. An AUROC statistic of 0.76 can be used for identifying when a patient was going to have severe grade CRS when only incorporating HR, RR, SpO2, and body temperature-related features. This supports patients being continuously and remotely monitored for severe CRS.

Clinical parameters beyond basic vital signs, which can be incorporated episodically, will enhance model accuracy. GCS and blood pressure enhanced the predictive performance in this exemplary cohort. In all six of our model types, there is a significant difference (p<0.05) in our models' discriminatory accuracy depending on the features incorporated. The discrimination was the highest using all features, vitals, and GCS scores, and higher using the full complement of vitals than HR, RR, SpO2 and body temperature alone. Further, when looking at the top 10 features with respect to feature importance for practical models, verbal GCS and SBP appear repeatedly. Models can benefit from periodically attending caregivers by incorporating this clinical information. However, the examples are not limited to the feature importance values or features shown in FIG. 20. In other examples, features could have different importance and/or include other features not shown in FIG. 20.

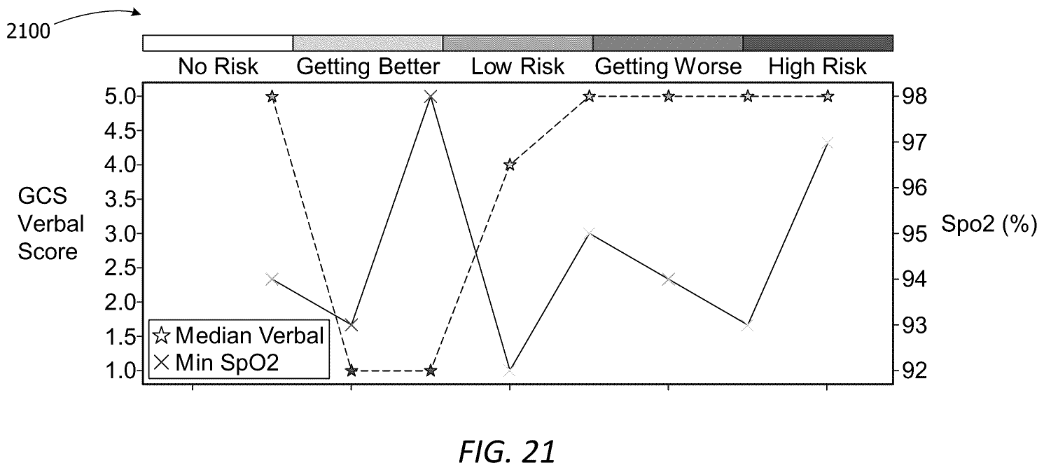

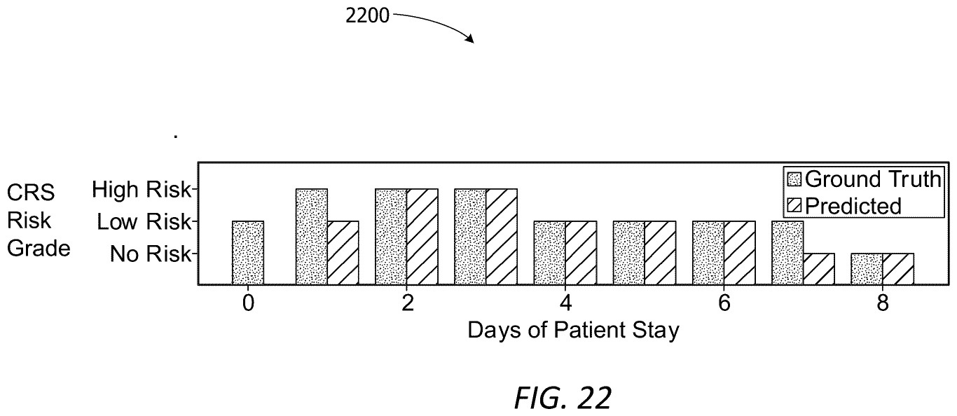

Referring to FIG. 21, an exemplary line graph 2100 illustrating an example patient journey where ML models predict CRS gradation levels or predict relative change in CRS gradations are shown. FIG. 22 is an exemplary bar graph 2200 illustrating an example patient journey where ML models predict CRS gradation levels or predict relative change in CRS gradations. The CRS gradations in the examples shown in FIG. 21 and FIG. 22 include no CRS, mild, moderate, or severe. The predicted relative change in RS gradations in this example include low, mild, or severe, worsening or improving from the previous predictions.

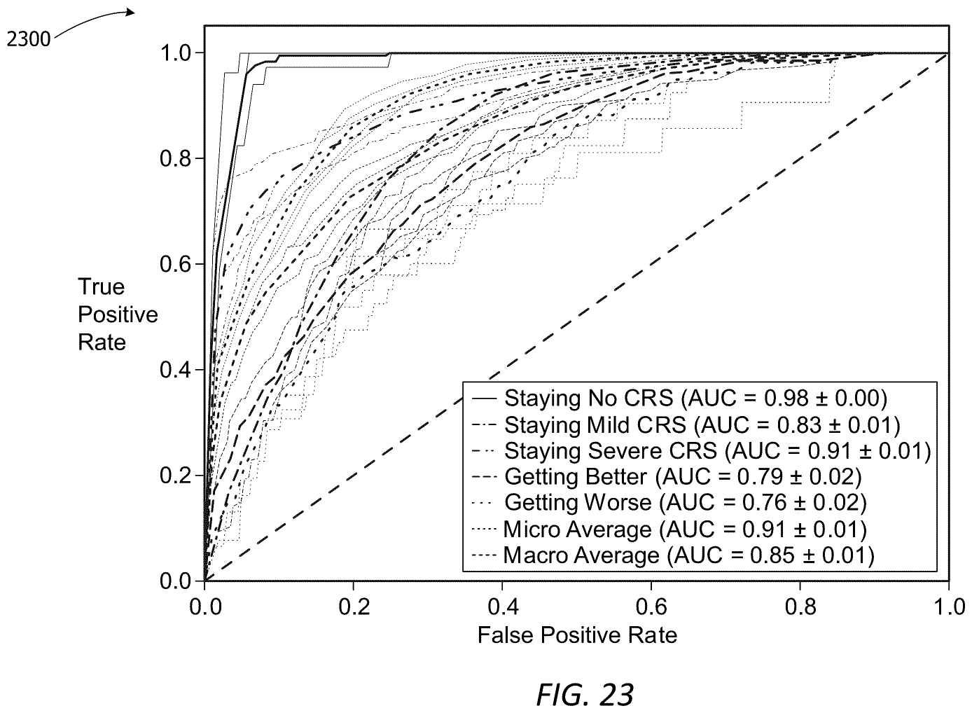

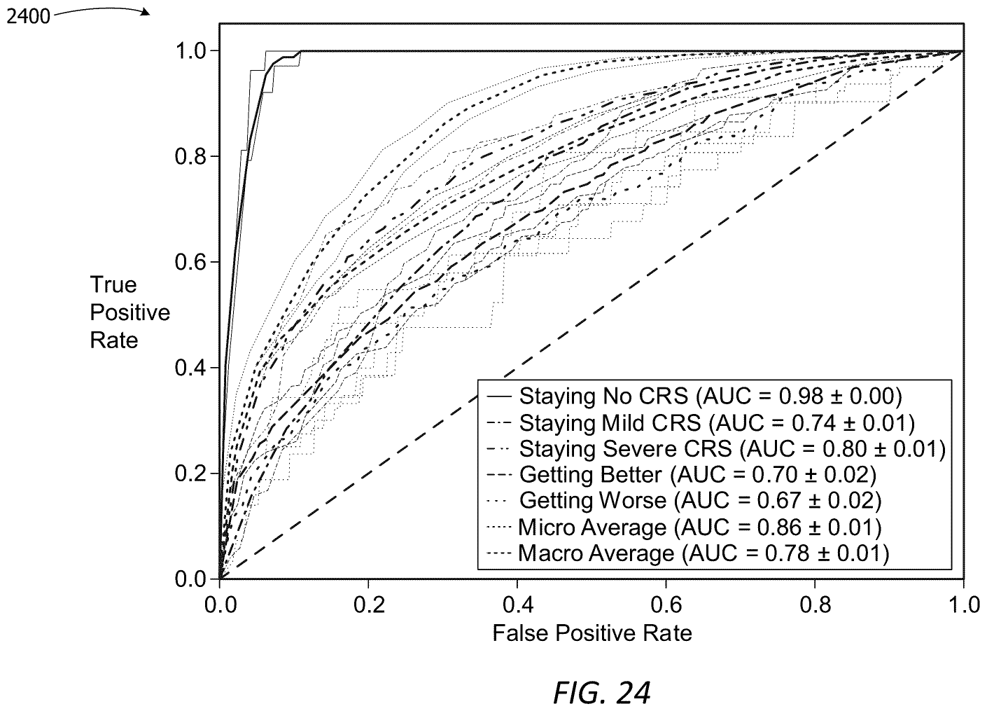

FIG. 23 is an exemplary graph 2300 illustrating ROC performances of ML models predicting relative changes in CRS gradations. FIG. 24 is an exemplary graph 2400 illustrating ROC performances of ML models predicting five classes of relative changes in CRS gradations when incorporating vital signs. In FIG. 23 and FIG. 24, the ROC performance of ML models predict five classes of relative change in CRS gradations taking all features as inputs.

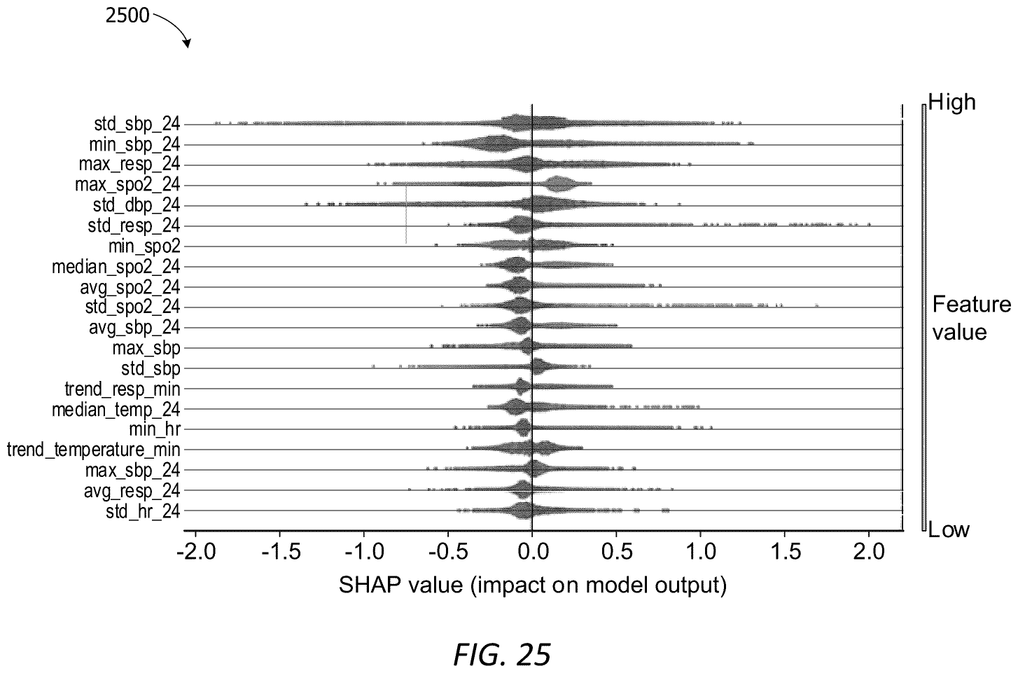

FIG. 25 is an exemplary graph 2500 illustrating feature importance from ML models predicting five classes of relative change in CRS gradations. FIG. 25 illustrates the ROC performances of ML models predicting five classes of relative changes in CRS gradations taking only vitals related features as inputs, which is a practical use case of remote patient monitoring using one or more wearable or vitals monitors. FIG. 21 is an exemplary illustration of feature importance from ML models predicting five classes of relative changes in CRS gradations taking only vitals related features as inputs. However, the examples are not limited to the feature importance shown in FIG. 25. In other examples, different features having different importance could be included.

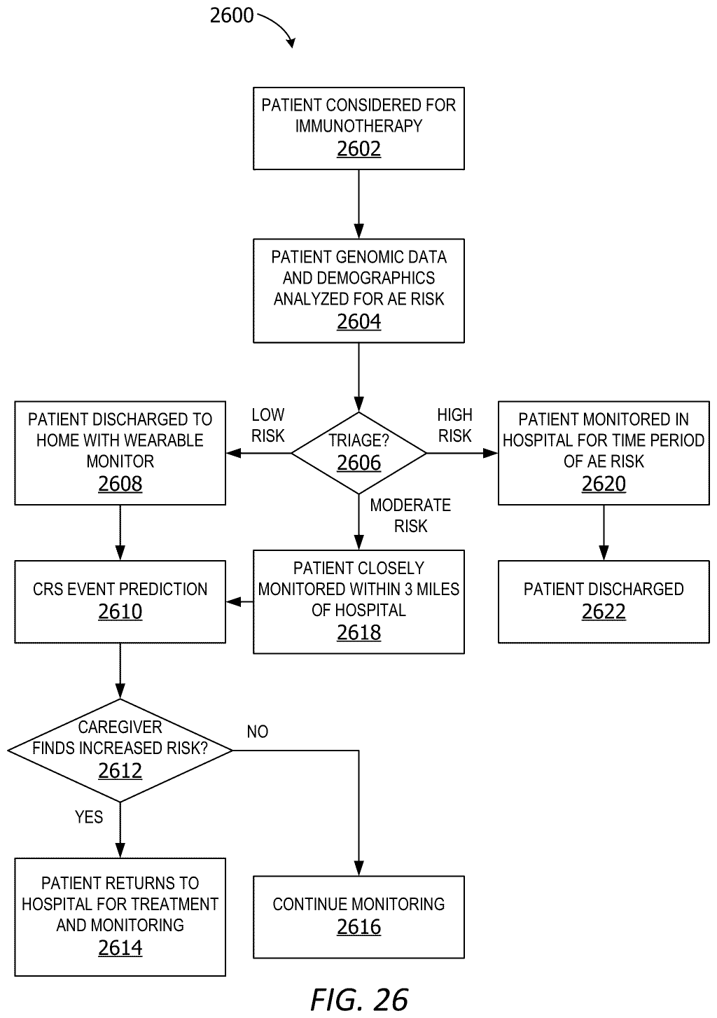

Referring now to FIG. 26, an exemplary flowchart 2600 shows an improved patient care pathway made possible by, facilitated by, coupled with, or otherwise associated with the CRS onset, grading, and deterioration prediction system. In some examples, a patient is considered for immunotherapy at 1202. The patient genomic data and demographics are analyzed for AE risk at 2604. In other words, when a patient is considered for a particular treatment with a particular adverse event profile, the patient's risk for experiencing a severe adverse event is assessed based on patient's historic data.

A triage determination is made based on the analysis results at 2606. If the patient is low risk at 2606, the patient is discharged with a wearable monitor at 2608. This enables the patient to return home or to any other resident care facility. Thus, if the patient is low risk, then the patient is able to be monitored in a remote setting for adverse event onset using the CRS onset, grading, and deterioration prediction system. The system generates a CRS event prediction at 2610. A determination is made whether the caregiver finds an increased risk or need to treat the patient at 2612. If yes, the patient returns to the hospital or other treatment facility for treatment and monitoring until the AE window closes at 2614.

If the patient is deemed to be moderate risk 2618, then the patient is monitored more closely within three miles or less distance to the hospital or other medical facility using the CRS onset, grading, and deterioration prediction system at 2610. A determination is made whether the caregiver finds an increased risk or need to treat the patient at 2612. If yes, the patient returns to the hospital or other treatment facility for treatment and monitoring until the AE window closes at 2614.

If the patient is high risk at 2606, the patient is monitored in the hospital using the CRS onset, grading, and deterioration prediction system at 2620. The patient is discharged from the hospital when the AE is reversed or mitigated sufficiently at 2622.

In some examples, the system provides notifications to monitoring caregivers to aid in their decision-making regarding changing the patients monitoring and whether or not the patient requires treatment for an adverse event. The system can incorporate different devices and allow for different levels of monitoring depending on the patient. The triage scheme for the patient could involve fewer or more levels. There could be any combination of remote or clinical monitoring options.

### ADDITIONAL EXAMPLES

In some examples, the system provides for cytokine release syndrome (CRS) event prediction coupled with a patient care pathway for patient's at-risk for iatrogenic CRS. The system enables prediction of CRS severity and associated deterioration in patients surgically treated for neoplasms. In other examples, CRS onset could be predicted for patients receiving immunotherapy treatments. CRS is a noninfectious systemic inflammatory response caused by surgeries, cancer therapies, or other treatments. The specific definition of this condition in the examples dictates true positives and true negatives, how it is separated from other SIRS conditions, etc. Prediction of CRS adverse events and their severity remains a challenge that needs to be solved. The earlier a patient is known to be having a CRS episode, identified by specific standards, the lower the costs and the better the outcomes can be for that patient. For example, oncology patients who are receiving treatment that put them at risk for an iatrogenic CRS can be monitored safely with the coupled care pathway and system of the examples.

The system in other examples is a flexible and adaptable system capable of utilizing diverse input features available from a plurality of different data sources to generate CRS event onset predictions and CRS outcome predictions. The system is also capable of operating in diverse environment, such as, but not limited to, hospitals, clinics, nursing homes, rehabilitation centers. The system can also be utilized in non-clinical settings, such as patient residences. Patient residences can include a private single-family home as well as resident care facilities, such as retirement homes and assisted living facilities.

In another example, a system is provided by which oncology patients receiving treatment and at-risk for iatrogenic cytokine release syndrome are safely monitored. In one care pathway, a patient is considered for a certain treatment. The patient is entered in continuous monitoring system that incorporates all indicated data types. The risk of the patient developing CRS due to the treatment is determined and the patient is triaged accordingly. The patient receives treatment. The patient is discharged to appropriate monitoring conditions based on triage and previous patients. The patient is monitored according to the system described in some examples of the described herein. The patient is monitored for likelihood of CRS onset. If CRS is not likely to onset, monitoring continues. If CRS is likely to onset, then a caregiver is alerted. If CRS has onset, then the CRS graded and the potential for deterioration is predicted. The caregiver is alerted with these data. The patient is monitored until AE resolves or window of risk passes.

The system in other examples generates CRS events information, including CRS onset timing, CRS grades and the relative changes in deterioration or amelioration output by the models directly without explicitly indicating the timing of onset. The CRS event data output by the system can include any one or combination of onset, grades, or deterioration related outputs. Different possible outcomes/outputs can include probability, category, time period, improvement, or deterioration.

The output of the system can include a single output as well as two or more (multiple) outputs. The output can include a prediction of CRS onset, a notification to a user, etc. The notification can include a text message, an email, a popup on a UI, or any other type of notification. The system can also trigger an alert, such as a visual or audible alert. An alert can include a flashing light on a display or a sound, such as an alarm, bell, beeping, or other alert sound. An alert can also include haptic alerts, as well as any other type of alert to medical personnel or other caregivers.

In other examples, a computing device provides a single point that runs the models and displays alerts to a caregiver directly with no network. In other examples, the computing device interfaces with a network and provides alerts to other devices.

Alternatively, or in addition to the other examples described herein, examples include any combination of the following:
obtaining patient-related health data for a monitored patient, the patient-related health data comprising vital signs data obtained from a set of sensor devices associated with the monitored patient and user-provided data associated with the monitored patient;
analyzing the patient-related health data using a trained CRS prediction manager, including a trained machine learning prediction model;
generating a CRS prediction based on analysis of the patient-related health data, the CRS prediction comprising a probability of a CRS event occurring within a predetermined time-period after generation of the CRS prediction;
providing a notification if a probability of the CRS event exceeds a threshold probability indicating onset of the CRS event is likely to occur within the predetermined time-period;
wherein the clinician provided data further comprises Glasgow coma scale (GCS) data associated with the monitored patient;
selecting the machine learning prediction model from a plurality of machine learning prediction models having a training cohort most similar to the monitored patient;
predicting a grade indicating a severity of the CRS event, wherein the grade is selected from a set of grades;
generating a predicted probability of the CRS event outcome, wherein the probability indicates whether a patient condition is likely to improve or decline within the predetermined time-period;
predicting CRS events in patients treated with a novel immunotherapy for a certain cancer type;
generating predictions for occurrence of CRS events within a next twenty-four-hour time-period or the next eight-hour time-period;
a set of sensor devices generating vital signs data associated with a monitored patient;
calculate a probability of CRS event onset using a trained machine learning (ML) model and patient-related health data, the patient-related health data comprising the vital signs data and user-provided health data associated with the monitored patient;
generate a CRS prediction associated with the monitored patient using the calculated probability of CRS event onset;
provide a notification including the generated CRS prediction, the notification comprising the calculated probability of the CRS event onset occurring within a predetermined time-period;
a user interface (UI) device, wherein the notification is provided to a user via the UI device;
generate a visualization representing the generated CRS prediction;
present the visualization to a user via a UI device;
aggregate user-provided data obtained from a plurality of sources, wherein the aggregated user-provided data includes sensor data obtained from the set of sensor devices and user-provided data provided by at least one of the monitored patients and a clinician;
analyse the aggregated user-provided data by the trained ML model to generate the CRS prediction;
obtaining patient-related health data for a monitored patient, the patient-related health data comprising vital signs data obtained from a set of sensor devices associated with the monitored patient and user-provided data associated with the monitored patient;
analyzing the patient-related health data using a trained CRS prediction manager system, including a trained machine learning prediction model; and
generating a CRS prediction using the patient-related health data, the CRS prediction comprising a probability of a CRS event onset occurring within a predetermined time-period after generation of the CRS prediction;
generating a notification, including the generated CRS prediction, the notification comprising the probability of the CRS event onset within the predetermined time-period; and
presenting the notification via a user interface (UI) device;
generate a prediction report including the generated CRS prediction associated with the monitored patient, the notification comprising the calculated probability of the CRS event onset occurring within a predetermined future time-period;
wherein a CRS event includes an onset, grade and/or deterioration prediction;
the generated CRS prediction comprising at least one of a probability of CRS onset, a grading of the CRS event and a deterioration prediction;
present the prediction report to a user via a UI device;
generate predictions of CRS-related adverse event onset time and associated alerts to aid in determining when a patient monitored in an outpatient setting is transported back to hospital for treatment;
generate CRS grading or severity-related prediction that aids in determining when a patient is transported from an outpatient setting to an in-patient setting for treatment;
generate predictions of times or the rate at which a patient is likely to deteriorate from a CRS-related adverse event in an outpatient setting and associated alerts to aid in caregivers making an assessment on whether to transport a patient back to an in-hospital setting; and
generate a predicted grade indicating severity of a CRS event, wherein the predicted grade comprises at least one of a mild grade, a moderate grade, or a severe grade.

At least a portion of the functionality of the various elements in FIG. 1, FIG. 2, FIG. 3, FIG. 4, FIG. 5, FIG. 6, and FIG. 7 can be performed by other elements in FIG. 1, FIG. 2, FIG. 3, FIG. 4, FIG. 5, FIG. 6 and FIG. 7, or an entity (e.g., processor 304, web service, server, application program, computing device, etc.) not shown in FIG. 1, FIG. 2, FIG. 3, FIG. 4, FIG. 5, FIG. 6, and FIG. 7.

In some examples, the operations illustrated in FIG. 7, FIG. 8 and FIG. 9 can be implemented as software instructions encoded on a computer-readable medium, in hardware programmed or designed to perform the operations, or both. For example, aspects of the disclosure can be implemented as a system on a chip or other circuitry including a plurality of interconnected, electrically conductive elements.

While the aspects of the disclosure have been described in terms of various examples with their associated operations, a person skilled in the art would appreciate that a combination of operations from any number of different examples is also within scope of the aspects of the disclosure.

The term “Wi-Fi” as used herein refers, in some examples, to a wireless local area network using high frequency radio signals for the transmission of data. The term “BLUETOOTH®” as used herein refers, in some examples, to a wireless technology standard for exchanging data over short distances using short wavelength radio transmission. The term “NFC” as used herein refers, in some examples, to a short-range high frequency wireless communication technology for the exchange of data over short distances.

While no personally identifiable information is tracked by aspects of the disclosure, examples have been described with reference to data monitored and/or collected from the users. In some examples, notice is provided to the users of the collection of the data (e.g., via a dialog box or preference setting) and users are given the opportunity to give or deny consent for the monitoring and/or collection. The consent can take the form of opt-in consent or opt-out consent.

### Exemplary Operating Environment

Exemplary computer-readable media include flash memory drives, digital versatile discs (DVDs), compact discs (CDs), floppy disks, and tape cassettes. By way of example and not limitation, computer-readable media comprise computer storage media and communication media. Computer storage media include volatile and nonvolatile, removable, and non-removable media implemented in any method or technology for storage of information such as computer-readable instructions, data structures, program modules and the like. Computer storage media are tangible and mutually exclusive to communication media. Computer storage media are implemented in hardware and exclude carrier waves and propagated signals. Computer storage media for the purposes of this disclosure are not signals per se. Exemplary computer storage media include hard disks, flash drives, and other solid-state memory. In contrast, communication media typically embody computer-readable instructions, data structures, program modules, or the like, in a modulated data signal such as a carrier wave or other transport mechanism and include any information delivery media.

Although described in connection with an exemplary computing system environment, examples of the disclosure are capable of implementation with numerous other special purpose computing system environments, configurations, or devices.

Examples of well-known computing systems, environments, and/or configurations that can be suitable for use with aspects of the disclosure include, but are not limited to, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. Such systems or devices can accept input from the user in any way, including from input devices such as a keyboard or pointing device, via gesture input, proximity input (such as by hovering), and/or via voice input.

Examples of the disclosure can be described in the general context of computer-executable instructions, such as program modules, executed by one or more computers or other devices in software, firmware, hardware, or a combination thereof. The computer-executable instructions can be organized into one or more computer-executable components or modules. Generally, program modules include, but are not limited to, routines, programs, objects, components, and data structures that perform tasks or implement abstract data types. Aspects of the disclosure can be implemented with any number and organization of such components or modules. For example, aspects of the disclosure are not limited to the specific computer-executable instructions, or the specific components or modules illustrated in the figures and described herein. Other examples of the disclosure can include different computer-executable instructions or components having more functionality or less functionality than illustrated and described herein.

In examples involving a general-purpose computer, aspects of the disclosure transform the general-purpose computer into a special-purpose computing device when configured to execute the instructions described herein.

The order of execution or performance of the operations in examples of the disclosure illustrated and described herein is not essential, unless otherwise specified. That is, the operations can be performed in any order, unless otherwise specified, and examples of the disclosure can include additional or fewer operations than those disclosed herein. For example, it is contemplated that executing or performing an operation before, contemporaneously with, or after another operation is within the scope of aspects of the disclosure.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e., “one or the other but not both”) when preceded by terms of exclusivity, such as “either”, “one of”, “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A system for cytokine release syndrome (CRS) event prediction coupled with a patient care pathway for patient's at-risk for CRS, the system comprising:
a set of sensor devices generating physiological data associated with a monitored patient;
a computer-readable medium storing instructions that are operative upon execution by a processor to:
calculate one or more probabilities of a CRS event using a plurality of machine learning (ML) models trained on patient-related health data, the patient-related health data comprising the physiological data, patient-reported outcomes data, and user-provided health data associated with the monitored patient;
generate a CRS prediction associated with the monitored patient using the calculated one or more probabilities of the CRS event; and
provide a notification including the generated CRS prediction, the notification comprising the calculated one or more probabilities of the CRS event occurring within a predetermined time-period.

2. The system of claim 1, wherein the instructions are further operative to:
generate a predicted grade indicating severity of the CRS event, wherein the predicted grade is selected from a plurality of grades.

3. The system of claim 1, wherein the instructions are further operative to:
generate a predicted probability of the CRS event, wherein the predicted probability indicates whether a condition of the monitored patient is likely to improve or decline within the predetermined time-period.

4. The system of claim 1, wherein the instructions are further operative to:
generate a visualization representing the generated CRS prediction, wherein the generated CRS prediction comprises at least one of a probability of CRS onsetting within a predefined window of time, a grading of the CRS event and a risk score of the patient deteriorating; and
present the visualization to a user via a user interface (UI) device.

5. The system of claim 1, wherein the instructions are further operative to:
aggregate user-provided health data obtained from a plurality of sources, wherein the aggregated user-provided data includes sensor data obtained from the set of sensor devices and user-provided data provided by at least one of the monitored patient and a clinician; and
analyze the aggregated user-provided data by the trained ML model to generate the CRS prediction.

6. The system of claim 1, wherein the instructions are further operative to:
generate risk scores or classes that aid in triage of a patient in the time preceding or immediately following infusion to dictate the necessary time and level of monitoring the patient may require in-hospital and or in an outpatient setting following infusion to ensure timely treatment of adverse events.

7. The system of claim 1, wherein the instructions are further operative to:
generate predictions of CRS-related adverse event onset time and associated notifications to aid in determining when a patient monitored in an outpatient setting is transported back to hospital for treatment;
generate CRS grading or severity-related prediction that aids in determining when a patient is transported from an outpatient setting to an in-patient setting for treatment; and
generate predictions of times or rate at which a patient is likely to deteriorate from a CRS-related adverse event in an outpatient setting and associated notifications to aid in caregivers making an assessment on whether to transport a patient back to an in-hospital setting.

8. A computational method for CRS event prediction, the method comprising:
calculating a probability of a CRS event using a plurality of machine learning (ML) models trained on patient-related health data, the patient-related health data comprising physiological data, patient-reported outcomes data, and user-provided health data associated with a monitored patient;
generating a CRS prediction associated with the monitored patient using the calculated probability of the CRS event; and
providing a notification including the generated CRS prediction, the notification comprising the calculated probability of the CRS event occurring within a predetermined time-period.

9. The computational method of claim 8, wherein the patient-related health data further comprises Glasgow coma scale (GCS) data associated with the monitored patient.

10. The computational method of claim 8, further comprising:
generating risk scores or classes that aid in triage of a patient in the time preceding or immediately following infusion to dictate the necessary time and level of monitoring the patient may require in-hospital and or in an outpatient setting following infusion to ensure timely treatment of adverse events.

11. The computational method of claim 8, further comprising:
predicting a grade indicating a severity of the CRS event, wherein the grade is selected from a set of grades.

12. The computational method of claim 8, further comprising:
generating a predicted probability of the CRS event, wherein the predicted probability indicates whether a condition of a monitored patient is likely to improve or decline within the predetermined time-period.

13. The computational method of claim 8, wherein the notification further comprises the generated CRS prediction and the probability of the CRS event occurring within the predetermined time-period; and
presenting the notification via a user interface (UI) device.

14. The computational method of claim 8, further comprising:
generating a visualization representing the CRS prediction, wherein the generated CRS prediction comprises at least one of a probability of CRS onsetting within a predefined window of time, a grading of the CRS event and a risk score of the patient deteriorating; and
presenting the visualization to a user via a UI device.

15. One or more computer storage devices having computer-executable instructions stored thereon, which, upon execution by a computer, cause the computer to perform operations comprising:
calculate a probability of a CRS event using a trained ML model and patient-related health data, the patient-related health data comprising physiological data and user-provided health data associated with a monitored patient;
generate a CRS prediction associated with the monitored patient using the calculated probability of the CRS event;
generate a prediction report including the generated CRS prediction associated with the monitored patient, the prediction report comprising the calculated probability of the CRS event occurring within a predetermined time-period; and
present the prediction report to a user via a UI device.

16. The one or more computer storage devices of claim 15, wherein the operations further comprise:
generate a predicted grade indicating severity of the CRS event, wherein the predicted grade comprises at least one of a mild grade, a moderate grade, or a severe grade.

17. The one or more computer storage devices of claim 15, wherein the operations further comprise:
generate risk scores or classes that aid in triage of a patient in the time preceding or immediately following infusion to dictate the necessary time and level of monitoring the patient may require in-hospital and or in an outpatient setting following infusion to ensure timely treatment of adverse events.

18. The one or more computer storage devices of claim 15, wherein the operations further comprise:
generate a visualization representing the generated CRS prediction; and
present the visualization to a user via the UI device.

19. The one or more computer storage devices of claim 15, wherein the operations further comprise:
aggregate user-provided health data obtained from a plurality of sources, wherein the aggregated user-provided data includes sensor data obtained from a set of sensor devices and user-provided data provided by at least one of the monitored patients and a clinician; and
analyze the aggregated user-provided data by the trained ML model to generate the CRS prediction.

20. The one or more computer storage devices of claim 15, wherein the operations further comprise:
generating a predicted probability of the CRS event, wherein the predicted probability indicates whether a condition of the monitored patient is likely to improve or decline within the predetermined time-period.
